(19) United States
(12) Patent Application Publication
(10) Pub. No.: US 2005/0277600 A1
(43) Pub. Date: Dec. 15, 2005

(54) Compositions and methods of use of dimer digallates

(75) Inventors: Harold H. Schmitz, Bethesda, MD (US); Catherine L. Kwik-Uribe, Stroudsburg, PA (US); Mark A. Kelm, East Stroudsburg, PA (US); John F. Hammerstone JR., Nazareth, PA (US); Leo J. Romanczyk, Hackettstown, NJ (US)

Correspondence Address: NADA JAIN, P.C., 560 White Plains Road, Suite 460, Tarrytown, NY, 10591, US

(73) Assignee: Mars, Incroporated, McLean, VA (US)

(21) Appl. No.: 11/153,698

(22) Filed: Jun. 14, 2005

Related U.S. Application Data
(60) Provisional application No. 60/579,303, filed on Jun. 14, 2004.

(51) Int. Cl. A61K 31/7048; A61K 31/353

(52) U.S. Cl. 514/27; 514/456

(57) ABSTRACT

The invention relates to compositions, such as pharmaceuticals, foods, food additives, or dietary supplements, containing dimer digallates, and methods of use thereof, for prophylactic or therapeutic treatment of a human or a veterinary animal to treat or prevent NO-responsive health conditions, treat hypertension, cardiovascular disease, coronary artery disease, diabetes, cognitive dysfunction or disorder and/or vascular circulation disorders, prevent or reduce the risk of heart attack, stroke, congestive heart failure and/or kidney failure, or to improve blood flow, for example renal blood flow. The composition may optionally contain an additional NO modulating agent and/or a vascular-protective or therapeutic agent, or may be administered in combination with such an agent.

This application claims the benefit, under 35 USC Section 119, of the U.S. Provisional Appl. No. 60/579,303 filed Jun. 14, 2004, the disclosure of which is hereby incorporated herein by reference.

### FIELD OF THE INVENTION

The invention relates to compositions containing procyanidin dimers and/or dimer digallates, and methods of use thereof, for prophylactic or therapeutic treatment of a human or a veterinary animal.

### BACKGROUND OF THE INVENTION

Polyphenols are an incredibly diverse group of compounds (Ferriera et al., Tetrahedron, 48:10, 1743-1803, 1992). They widely occur in a variety of plants, some of which enter into the food chain. In some cases they represent an important class of compounds for the human diet. Although some of the polyphenols are considered to be non-nutritive, interest in these compounds has arisen because of their possible beneficial effects on health. For instance, quercetin (a flavonoid) has been shown to possess anticarcinogenic activity in experimental animal studies (Deshner et al., Carcinogenesis, 7:1193-1196, 1991: and Kato et al., Carcinogenesis, 4, 1301-1305 1983). (+)-catechin and (−)-epicatechin (flavan-3-ols) have been shown to inhibit Leukemia virus reverse transcriptase activity (Chu et al., J. of Natural Prod., 55:2, 179-183, 1992). Nobotanin (an oligomeric hydrolyzable tannin) has also been shown to possess anti-tumor activity (Okuda et al., presented at the XVIth International Conference of the Groupe Polyphenols, Lisbon, Portugal, Jul. 13-16, 1992). Procyanidin oligomers have been reported by the Kikkoman Corporation for use as antimutagens (JP 04190774A, Jul. 7, 1992).

Nitric oxide (NO) is known to inhibit platelet aggregation, monocyte adhesion and chemotaxis, and proliferation of vascular smooth muscle tissue which are critically involved in the process of atherogenesis. The concentration of NO can be reduced in atherosclerotic tissues due to its reaction with oxygen free radicals. The loss of NO due to these reactions leads to increased platelet and inflammatory cell adhesion to vessel walls to further impair NO mechanisms of relaxation. In this manner, the loss of NO may promote atherogenic processes, leading to progressive disease states.

Hypertension is a condition where the pressure of blood as it circulates within the blood vessels is higher than normal. When the systolic pressure exceeds 150 mm Hg or the diastolic pressure exceeds 90 mm Hg for a sustained period of time, damage is done to the body. Hypertension is a leading cause of vascular diseases, including stroke, heart attack, heart failure, and kidney failure. For example, excessive systolic pressure can rupture blood vessels anywhere. In cases when a rupture occurs within the brain, a stroke results. Hypertension can also cause thickening and narrowing of the blood vessels which can lead to atherosclerosis. Elevated blood pressure can also force the heart muscle to enlarge as it works harder to overcome the elevated resting (diastolic) pressure when blood is expelled. This enlargement can eventually produce irregular heart beats or heart failure. Hypertension is called the “silent killer” because it causes no symptoms and can only be detected when blood pressure is checked.

The regulation of blood pressure is a complex event where one mechanism involves the expression of constitutive Ca+2/calmodulin dependent form of nitric oxide synthase (NOS), known as endothelial nitric oxide synthase or eNOS. NO produced by this enzyme produces smooth muscle relaxation in the vessel (dilation), which lowers the blood pressure. When circulating concentrations of NO are reduced, either because production is blocked by an inhibitor or in pathological states, such as atherosclerosis, the vascular muscles do not relax to the appropriate degree. The resulting vasoconstriction increases blood pressure and may be responsible for some forms of hypertension. Given the large number of people suffering from hypertension and related diseases and disorders of the vascular system, there is considerable interest in finding therapeutic ways to maintain the NO pool at its normal, healthy levels. Pharmacological agents capable of releasing NO, such as nitroglycerin or isosorbide dinitrate, remain mainstays of vasorelaxant therapy. Applicants have now discovered new compounds that are useful in treating and/or preventing NO-responsive diseases and disorders like hypertension.

### SUMMARY OF THE INVENTION

The invention relates to compositions comprising the dimer digallates, for example EC-(4β→8)-C digallate, C-(4α→8)-C digallate, C-(4β→8)-C digallate, and C-(4β→8)-EC digallate, (herein after referred to as “dimer digallates of the invention”), the dimers comprising an alpha linkage between the monomeric units, for example, 4α→8 linkage, such as C-(4α→8)-C, and methods of use thereof, for prophylactic or therapeutic treatment of a human or a veterinary animal. As used herein, EC means epicatechin and C means catechin.

In one aspect, the invention relates to a composition, such as a pharmaceutical, a food, a food additive, or a dietary supplement comprising an effective amount of the dimer digallate of the invention. The composition may optionally contain an additional NO modulating agent and/or a vascular (including cardiovascular)-protective or therapeutic agent, or may be administered in combination with such an agent.

Also within the scope of the invention are packaged products containing the above-mentioned compositions and a label and/or instructions for use to treat or prevent NO-responsive health conditions, treat or prevent hypertension, cardiovascular disease, coronary artery disease, diabetes (type I and type II), cognitive dysfunction or disorder and/or vascular circulation disorders, to prevent or reduce the risk of heart attack, stroke, congestive heart failure and/or kidney failure, or to improve blood flow, for example renal blood flow.

In another aspect, the invention relates to methods of use of the dimer digallates of the invention and/or the dimers comprising an alpha linkage between the monomeric units, for example, 4α→8 linkage, such as dimer C-(4α→8)-C to treat or prevent NO-responsive health conditions, hypertension, cardiovascular disease, coronary artery disease, diabetes (type I and type II), cognitive dysfunction or disorder and/or vascular circulation disorders (including those of the brain), prevent or reduce the risk of heart attack, stroke, congestive heart failure and/or kidney failure, or to improve blood flow, for example renal blood flow.

### BRIEF DESCRIPTION OF THE DRAWINGS

FIG. 1 represents the acute effects of test compounds on HUVEC NO production

FIGS. 2a and 2b represent the effects of test compounds on HUVEC NO production following a single dose administration at 10 μM (a) or 1 μM (b) and a 24 hour incubation.

FIG. 3 represents HUVEC NO production following 5 subsequent 24 hour treatments.

FIG. 4 represents the effects of test compounds on HUVEC NO production induced by a single 10 μM dose followed by a 24 hour incubation.

FIG. 5 represents the effects of test compounds on HUVEC NO production induced by a single 1 μM dose followed by a 24 hour incubation.

FIG. 6 represents HUVEC NO production following 5 subsequent 24 hour treatments.

FIG. 7 represents HUVEC-mediated MTT reduction following 5 subsequent 24 hour treatments.

FIG. 8 represents the effects of test compounds on pre-contracted aortic rings.

FIG. 9 represents dose-dependent relaxation mediated by test compounds in pre-contracted aortic rings.

FIG. 10 represents the effects of test compounds on pre-contracted aortic rings.

FIG. 11 represents dose-dependent relaxation mediated by test compounds in pre-contracted aortic rings.

### DETAILED DESCRIPTION

All patents, patent applications and references cited in this application are hereby incorporated herein by reference. In case of any inconsistency, the present disclosure governs.

The invention relates to compositions comprising an effective amount of a dimer digallate, or a pharmaceutically acceptable salt or derivative thereof.

The dimer digallates of the invention are gallic acid esters of procyanidin dimers composed of two monomeric, flavan-3-ol units of the formula: 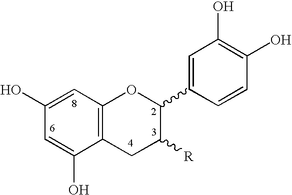
wherein R is O-gallate, R has either α or β stereochemistry, and monomeric units are connected via interflavan linkages 4→6 or 4→8, which have either α or β stereochemistry. Flavan-3-ol (monomeric) units may be (+)-catechin, (−)-epicatechin and their respective epimers (e.g. (−)-catechin and (+)-epicatechin). Thus, in certain embodiments, the dimer digallate of the invention has the following structure:

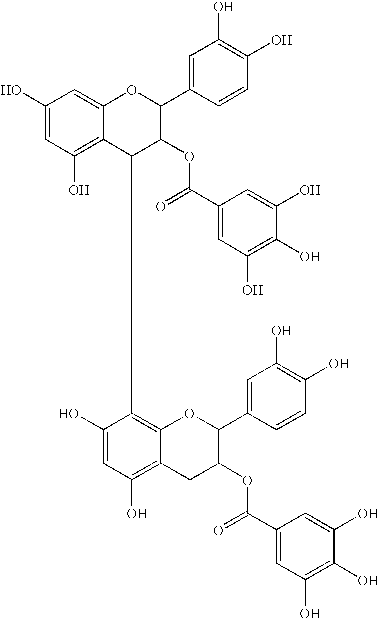

Also within the scope of the invention are derivatives of dimer digallates when any C-4, C-6 or C-8, which is not bonded to another monomeric unit, is derivatized with a sugar; which sugar may be optionally substituted with a phenolic moiety at any position, for instance, via an ester bond. The sugar can be selected from the group consisting of glucose, galactose, rhamnose, xylose, and arabinose. The sugar is preferably a mono-saccharide or di-saccharide. The phenolic moiety is selected from the group consisting of caffeic, cinnamic, coumaric, ferulic, gallic, hydroxybenzoic and sinapic acids.

In one embodiment, the dimer digallate is EC-(4β→8)-C digallate, or a pharmaceutically acceptable salt or derivative thereof. For example, the dimer is (−)EC-(4β→8)-(+)C digallate and has the following formula: 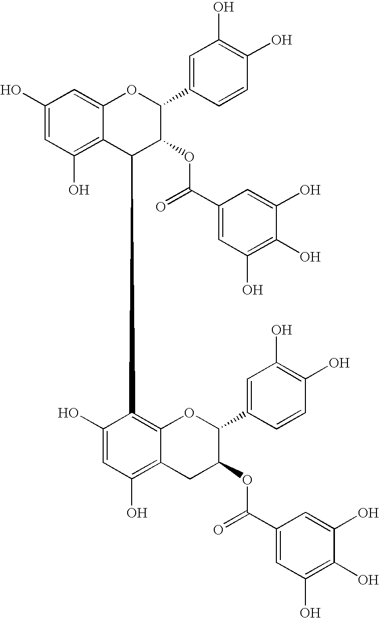
In another example, EC-(4β→8)-C digallate may be (−)EC-(4β→8)-(−)C digallate.

In another embodiment, the dimer digallate is C-(4α→8)-C digallate, or a pharmaceutically acceptable salt or derivative thereof. For example, the dimer is (+)C-(4α→8)-(+)C digallate and has the following formula:

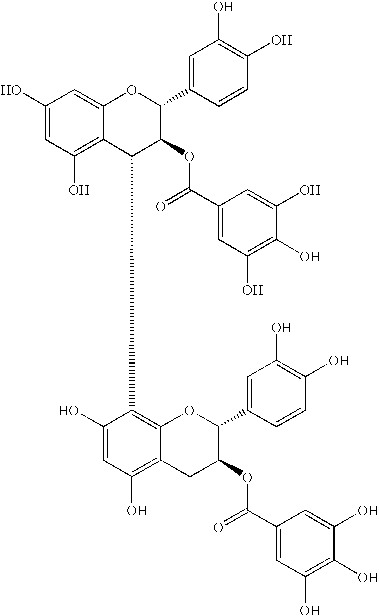

In yet another embodiment, the dimer digallate is C-(4β→8)-C digallate, or a pharmaceutically acceptable salt or derivative thereof. For example, the dimer is (+)C-(4β→8)-(+)C and has the following formula:

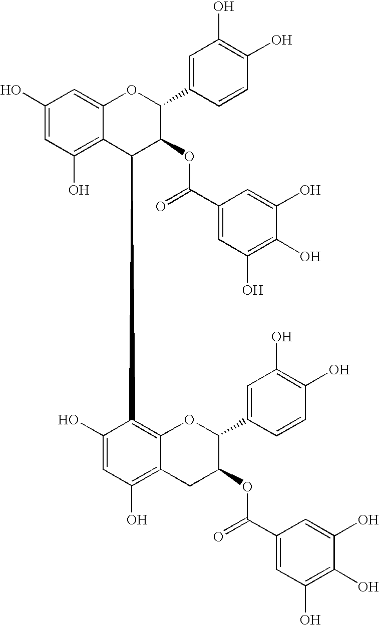

In yet another embodiment, the dimer digallate is C-(4β→8)-EC digallate, or a pharmaceutically acceptable salt or derivative thereof. For example, the dimer is (+)C-(4β→8)-(+)EC digallate and has the following formula:

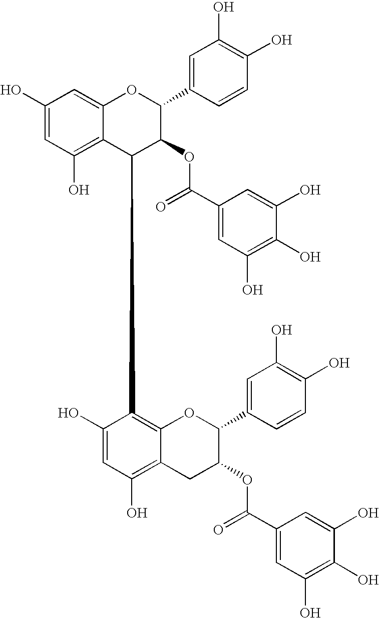

In other embodiments, the dimers comprising an alpha linkage between the monomeric units, for example, 4α→8 linkage, such as dimer C-(4α→8)-C, or a pharmaceutically acceptable salt or derivative thereof are within the scope of the invention. For example, the dimer is (+)C-(4α→8)-(+)C.

Dimer and dimer digallates of the invention may be of natural origin or synthetically prepared. They may be isolated from known plant sources using methods known in the art. For example, dimer digallates may be isolated from rhubarb as described in Nonaka et al., Chem. Pharm. Bull., 29: 2862-2870 (1981) and Kashavada et al., Chem. Pharm. Bull., 34: 4083-4091(1986), the relevant portions of each being hereby incorporated herein by reference. Rhubarb (Rhei Rhizoma) has been shown to contain dimer digallates. Commercial Rhubarb, treated to yield a low-molecular weight phenolic fraction, contains dimer digallate species along with a variety of other phenolic compounds.

The dimer and dimer digallates of the invention may also be synthetically prepared. For example, the two monomeric units may be coupled and esterified through the process described in U.S. Pat. No. 6,420,572 B1, which is hereby incorporated by reference. The process comprises coupling a protected monomer, having protected phenolic hydroxy groups, with a functionalized monomer. See Example 1.

The dimer and dimer digallates of the invention may be isolated and purified, i.e., they are separated from compounds with which they naturally occur (if the dimer digallate is of natural origin), or they are synthetically prepared, in either case such that the level of contaminating compounds (impurities) does not significantly contribute to, or detract from, the effectiveness of the dimer digallate. For example, an isolated and purified dimer digallate of the invention is separated from other procyanidins and polyphenols, with which it may occur in nature, to the extent achievable by the available commercially viable purification and separation techniques. The compounds may be substantially pure, i.e., they possess the highest degree of homogeneity achievable by the available purification, separation and/or synthesis technology. As used herein, a “substantially pure dimer digallate” is separated from other procyanidins and polyphenols to the extent technologically and commercially possible but may contain a mixture of several dimers. In other words, the phrase “isolated and purified dimer” refers primarily to one dimer, while a “substantially pure dimer” may encompass a mixture of dimers.

In some embodiments, the dimer and dimer digallates are at least 80% pure, preferably at least 85% pure, at least 90% pure, at least 95% pure, at least 98% pure, or at least 99% pure. Such compounds are particularly suitable for pharmaceutical applications.

As described below, the dimer and dimer digallates of the invention may be prepared in formulations and may be administered alone or in combination with other therapeutic agents to treat or prevent NO-responsive diseases or disorders. As such the dimer and dimer digallates of the invention may be used as anti-hypertensives, NO-preserving agents or NO-modulating agents. Dimers and dimer digallates of the invention may be administered in combination with vascular (including cardiovascular)-protective or therapeutic agents, such as other B-type procyanidins as well as A-type procyanidins. As such the compositions of the invention comprise admixtures.

Methods of Use

Any compound and/or composition described herein may be used to practice the methods described in the present application.

As used herein, an “NO-responsive disease or disorder” refers to a health condition which responds to treatment with NO. Examples of such conditions include, but are not limited, to NO-mediated or NO-dependent diseases and disorders, in which the pathology of the disease/disorder is caused by abnormal functioning of the NO pathway. Preferably the conditions include hypertension, cardiovascular disease, coronary artery disease, diabetes (type I and type II), cognitive dysfunction or disorder and/or vascular circulation disorders (including those of the brain), heart attack, stroke, congestive heart failure, kidney failure, and renal disease. Because high blood pressure increases the risk of heart attack, stroke, congestive heart failure, and kidney failure, dimer and dimer digallates which cause vasorelaxation can be utilized, alone or in combination with other vascular (including cardiovascular)-protective agents, to prevent these conditions. Particularly suitable subjects include subjects with high blood pressure in combination with diabetes, obesity, adverse lipid profile (e.g. high cholesterol levels) and/or smokers, in which patients the risk of heart attack and stroke increases several times. Generally, any subject having at least one of the cardiovascular disease risk factors (as recognized by the American Heart Association) may be treated as described herein.

As used herein, “treatment” means improving an existing medical condition, such as cardiovascular disease, for example by slowing down the disease progression, prolonging survival, reducing the risk of death, and/or providing a measurable improvement of disease parameters.

The term “preventing” means reducing the risks associated with developing a disease, including reducing the onset of the disease.

As used herein, the terms “vascular-protective or therapeutic agent” refers to an agent other than a dimer or dimer digallate of the invention which is effective to treat or protect vascular (including cardiovascular) system. Examples of such agents are anti-platelet therapy agents (e.g. COX inhibitors, such as aspirin; other B-type procyanidins, and A-type procyanidins); NO-modulating agents, cholesterol reducing agents (e.g. sterol, stanol).

Dimers and dimer digallates affect the nitric oxide (NO) pathway in endothelial cells helping preserve the NO pool. Without being bound by theory, the NO pool is preserved by inducing NO synthesis and/or decreasing NO degradation. The compounds also cause vasorelaxation of constricted blood vessels.

Thus, the invention relates to a method of treating or preventing an NO-responsive disease or disorder by administering to a subject in need thereof an effective amount of a dimer digallate, or a pharmaceutically acceptable salt or derivative thereof, wherein the subject is a human or a veterinary animal, which dimer digallate is a gallic acid ester of a procyanidin dimer composed of two monomeric, flavan-3-ol units of the formula: 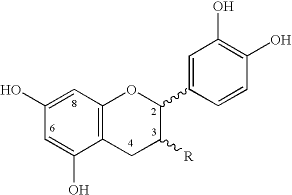
wherein R is O-gallate, R has either α or β stereochemistry, and monomeric units are connected via interflavan linkages 4→6 or 4→8, which have either α or β stereochemistry. Flavan-3-ol (monomeric) units may be (+)-catechin, (−)-epicatechin and their respective epimers (e.g. (−)-catechin and (+)-epicatechin).

In some embodiments, the method of treating or preventing an NO-responsive disease or disorder comprises administering to a subject in need thereof (a human or a veterinary animal) a therapeutically effective amount of a dimer digallate having the following formula, or a pharmaceutically acceptable salt or derivative thereof:

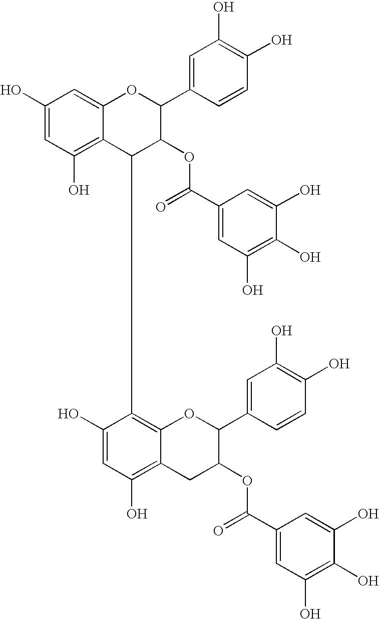

The dimer and dimer digallate for use in the methods of the invention may be isolated and purified or substantially pure. In some embodiments, the compounds described herein may be at least about 80% pure, at least about 85% pure, at least about 90% pure, at least 95% pure or at least 98% pure.

Also for use in the methods described herein are derivatives of dimer and dimer digallates when any C-4, C-6 or C-8, which is not bonded to another monomeric unit, is derivatized with a sugar; which sugar may be optionally substituted with a phenolic moiety at any position, for instance, via an ester bond. The sugar can be selected from the group consisting of glucose, galactose, rhamnose, xylose, and arabinose. The sugar is preferably a monosaccharide or di-saccharide. The phenolic moiety is selected from the group consisting of caffeic, cinnamic, coumaric, ferulic, gallic, hydroxybenzoic and sinapic acids.

In some embodiments, the invention relates to the following methods:

A method of treating or preventing an NO-responsive disease or disorder by administering to a subject in need thereof an effective amount of EC-(4β→8)-C digallate, for example (−)EC-(4β→8)-(+)C digallate, or a pharmaceutically acceptable salt or derivative thereof, wherein the subject is a human or a veterinary animal. The dimer digallate may be isolated and purified. In some embodiments, the above compound may be at least about 90% pure, at least 95% pure or at least 98% pure.

A method of treating or preventing an NO-responsive disease or disorder by administering to a subject in need thereof an effective amount of C-(4α→8)-C digallate, for example (+)C-(4α→8)-(+)C digallate, or a pharmaceutically acceptable salt or derivative, wherein the subject is a human or a veterinary animal. The dimer digallate may be isolated and purified. In some embodiments, the above compound may be at least about 90% pure, at least 95% pure or at least 98% pure.

A method of treating or preventing an NO-responsive disease or disorder by administering to a subject in need thereof an effective amount of C-(4β→8)-C digallate, for example (+)C-(4β→8)-(+)C digallate, or a pharmaceutically acceptable salt or derivative thereof, wherein the subject is a human or a veterinary animal. The dimer digallate may be isolated and purified. In some embodiments, the above compounds may be at least about 90% pure, at least 95% pure or at least 98% pure.

A method of treating or preventing an NO-responsive disease or disorder by administering to a subject in need thereof an effective amount of C-(4β→8)-EC digallate, for example (+)C-(4β→8)-(+)EC digallate, or a pharmaceutically acceptable salt or derivative thereof, wherein the subject is a human or a veterinary animal. The dimer digallate may be isolated and purified, or substantially pure. In some embodiments, the above compounds may be at least about 90% pure, at least 95% pure or at least 98% pure.

A method of treating or preventing an NO-responsive disease or disorder by administering to a subject in need thereof an effective amount of a dimer comprising an alpha linkage between the monomeric units, for example, 4α→8 linkage, such as a dimer C-(4α→8)-C, for example (+)C-(4α→8)-(+)C, or a pharmaceutically acceptable salt or derivative thereof, wherein the subject is a human or a veterinary animal. The dimer may be isolated and purified. In some embodiments, the above compound may be at least about 90% pure, at least 95% pure or at least 98% pure.

In certain embodiment, the invention provides for the following exemplary methods:

A method of treating hypertension (e.g. inducing vasorelaxation) by administering to a subject in need thereof an effective amount of a dimer digallate, or a pharmaceutically acceptable salt or derivative thereof, wherein the subject is a human or a veterinary animal, and which dimer digallate is a gallic acid ester of a procyanidin dimer composed of two monomeric, flavan-3-ol units of the formula: 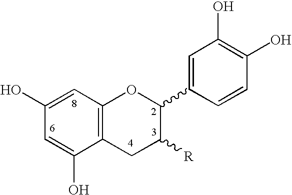
wherein R is O-gallate, R has either α or β stereochemistry, and monomeric units are connected via interflavan linkages 4→6 or 4→8, which have either α or β stereochemistry. Flavan-3-ol (monomeric) units may be (+)-catechin, (−)-epicatechin and their respective epimers (e.g. (−)-catechin and (+)-epicatechin). The dimer digallates of the invention may be isolated and purified or substantially pure. In some embodiments, the above compounds may be at least about 80% pure, at least about 85% pure, at least about 90% pure, at least 95% pure or at least 98% pure.

A method of treating hypertension (e.g. inducing vasorelaxation) by administering to a subject in need thereof an effective amount of a dimer digallate having the following formula, or a pharmaceutically acceptable salt or derivative thereof: 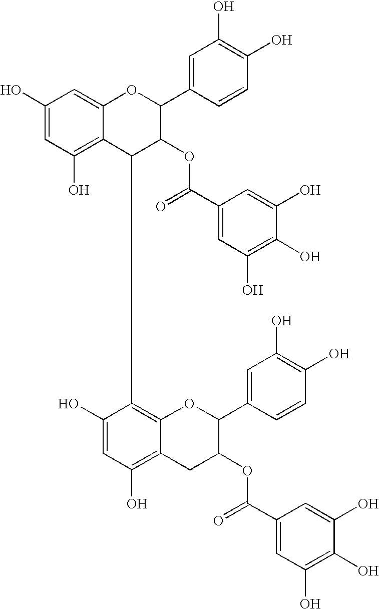
In some embodiments, the compounds described herein may be at least about 80% pure, at least about 85% pure, at least about 90% pure, at least 95% pure or at least 98% pure.

A method of treating hypertension (e.g. inducing vasorelaxation) by administering to a subject in need thereof an effective amount of EC-(4β→8)-C digallate, for example (−)EC-(4β→8)-(+)C digallate, or a pharmaceutically acceptable salt or derivative thereof, wherein the subject is a human or a veterinary animal. The dimer digallate may be isolated and purified. In some embodiments, the above compound may be at least about 90% pure, at least 95% pure or at least 98% pure.

A method of treating hypertension (e.g. inducing vasorelaxation) by administering to a subject in need thereof an effective amount of C-(4α→8)-C digallate, for example (+)C-(4α→8)-(+)C digallate, or a pharmaceutically acceptable salt or derivative thereof, wherein the subject is a human or a veterinary animal. The dimer digallate may be isolated and purified. In some embodiments, the above compound may be at least about 90% pure, at least 95% pure or at least 98% pure.

A method of treating hypertension (e.g. inducing vasorelaxation) by administering to a subject in need thereof an effective amount of C-(4β→8)-C digallate, for example (+)C-(4β→8)-(+)C digallate, or a pharmaceutically acceptable salt or derivative thereof, wherein the subject is a human or a veterinary animal. The dimer digallate may be isolated and purified. In some embodiments, the above compounds may be at least about 90% pure, at least 95% pure or at least 98% pure.

A method of treating hypertension (e.g. inducing vasorelaxation) by administering to a subject in need thereof an effective amount of C-(4β→8)-EC digallate, for example (+)C-(4β→8)-(−)EC digallate or a pharmaceutically acceptable salt or derivative thereof, wherein the subject is a human or a veterinary animal. The dimer digallate may be isolated and purified. In some embodiments, the above compounds may be at least about 90% pure, at least 95% pure or at least 98% pure.

A method of treating hypertension (e.g. inducing vasorelaxation) by administering to a subject in need thereof an effective amount of a dimer comprising an alpha linkage between the monomeric units, for example, 4α→8 linkage, such as dimer C-(4α→8)-C, for example (+)C-(4α→8)-(+)C, or a pharmaceutically acceptable salt or derivative thereof, wherein the subject is a human or a veterinary animal. The dimer may be isolated and purified. In some embodiments, the above compounds may be at least about 90% pure, at least 95% pure or at least 98% pure.

The effective amount for use in the above methods may be determined by a person of skill in the art using the guidance provided herein and general knowledge in the art. For example, the effective amount may be such as to achieve a physiologically relevant concentration in the body (e.g. blood) of a mammal. Such a physiologically relevant concentration may be at least about 10 nanomolar (nM), preferably at least about 20 nM, or at least about 100 nM, and more preferably at least about 500 nM. In one embodiment, at least about one micromole in the blood of the mammal, such as a human, is achieved. The compounds may be administered at from about 50 mg/day to about 1000 mg/day, preferably from about 100-150 mg/day to about 900 mg/day, and most preferably from about 300 mg/day to about 500 mg/day. However, amounts higher than stated above may be used.

The compounds may be administered acutely, or treatment/preventive administration may be continued as a regimen, i.e., for an effective period of time, e.g., daily, monthly, bimonthly, biannually, annually, or in some other regimen, as determined by the skilled medical practitioner for such time as is necessary. The administration may be continued for at least a period of time required to exhibit therapeutic/prophylactic effects. Preferably, the composition is administered daily, most preferably two or three times a day, for example, morning and evening to maintain the levels of the effective compounds in the body of the mammal. To obtain the most beneficial results, the composition may be administered for at least about 30, or at least about 60 days. These regiments may be repeated periodically. Based on the guidance provided herein and general knowledge in the art, a person of skill in the art can select compounds that are suitable for acute and or/chronic administration. For example, compounds that show effect after single dose administration and/or shortly after administration (e.g. two hours post administration) may be when quick, acute response is required. Compounds that show effect after repeated administration may be used accordingly. Thus, dosage forms adapted for such administration (e.g. acute, chronic) are within the scope of the invention.

Also within the scope of the invention are assays for determining a minimum therapeutically required dosage amount or an optimal dosage amount for use in the above therapeutic methods. Methods described in the examples, or any other dose response methods known to be predictive of compound effectiveness to treat hypertension and/or NO-responsive disease or disorder may be used. Dimers and dimer digallates may be tested in such assays. Dosage forms adapted to deliver at least a minimum therapeutically effective amount, or an optimal amount, are within the scope of the invention.

Compositions and Formulations

The compounds of the invention may be administered as a pharmaceutical, food, food additive or a dietary supplement.

As used herein a “food” is a material consisting essentially of protein, carbohydrate and/or fat, which is used in the body of an organism to sustain growth, repair and vital processes and to furnish energy. Foods may also contain supplementary substances such as minerals, vitamins and condiments. See Merriam-Webster's Collegiate Dictionary, 10th Edition, 1993. The term food includes a beverage adapted for human or animal consumption. As used herein a “food additive” is as defined by the FDA in 21 C.F.R. 170.3(e)(1) and includes direct and indirect additives. As used herein, a “pharmaceutical” is a medicinal drug. See Merriam-Webster's Collegiate Dictionary, 10th Edition, 1993. A pharmaceutical may also be referred to as a medicament. As used herein, a “dietary supplement” is a product (other than tobacco) that is intended to supplement the diet that bears or contains the one or more of the following dietary ingredients: a vitamin, a mineral, an herb or other botanical, an amino acid, a dietary substance for use by man to supplement the diet by increasing the total daily intake, or a concentrate, metabolite, constituent, extract or combination of these ingredients.

Pharmaceuticals containing the inventive compounds, optionally in combination with another vascular (including cardiovascular)-protective or therapeutic agent, may be administered in a variety of ways such as orally, sublingually, bucally, nasally, rectally, intravenously, parenterally and topically. A person of skill in the art will be able to determine a suitable mode of administration to maximize the delivery of the dimer and/or dimer digallates of the invention, optionally in combination with another vascular-protective or therapeutic agent. Thus, dosage forms adapted for each type of administration are within the scope of the invention and include solid, liquid and semi-solid dosage forms, such as tablets, capsules, gelatin capsules (gelcaps), bulk or unit dose powders or granules, emulsions, suspensions, pastes, creams, gels, foams, jellies or injection dosage forms. Sustained-release dosage forms are also within the scope of the invention and may be prepared as described in U.S. Pat. Nos. 5,024,843; 5,091,190; 5,082,668; 4,612,008 and 4,327,725, relevant portions of which are hereby incorporated herein by reference. Suitable pharmaceutically acceptable carriers, diluents, or excipients are generally known in the art and can be determined readily by a person skilled in the art. The tablet, for example, may comprise an effective amount of the dimer and/or dimer digallate containing composition and optionally a carrier, such as sorbitol, lactose, cellulose, or dicalcium phosphate.

The dietary supplement containing dimer and/or dimer digallate of the invention, and optionally another vascular-protective or therapeutic agent, may be prepared using methods known in the art and may comprise, for example, nutrient such as dicalcium phosphate, magnesium stearate, calcium nitrate, vitamins, and minerals.

Further within the scope of the invention is an article of manufacture such as a packaged product comprising the composition of the invention (e.g. a food, a dietary supplement, a pharmaceutical) and a label indicating the presence of, or an enhanced content of the inventive compounds or directing use of the composition for methods described herein.

Dosage forms adapted to deliver at least a minimum therapeutically effective amount, or an optimal therapeutic amount, are within the scope of the invention. Such amounts may be determined as described herein, and for example using guidance provided in the examples.

Also within the scope of the invention is an article of manufacture (such as a packaged product or kit) adapted for use in combination therapy comprising at least one container and at least one dimer and/or dimer digallate of the invention, or a pharmaceutically acceptable salt or derivatives thereof. The article of manufacture further comprises at least one additional agent, a vascular-protective or therapeutic agent (i.e., other than the dimer and/or dimer digallate of the invention, or a pharmaceutically acceptable salt or derivative thereof), which agent may be provided as a separate composition, in a separate container, or in admixture with the compound of the invention.

As described above, vascular-protective or therapeutic agents are effective to treat or protect vascular system (e.g. cardiovascular, brain). Examples of such agents are anti-platelet therapy agents (e.g. COX inhibitors, such as aspirin); NO-modulating agents, cholesterol reducing agents (e.g. sterol, stanol).

In certain embodiments, vascular-protective or therapeutic agents optionally administered with dimer digallates of the invention may be A-type procyanidins or other B-type procyanidins, as described below.

A-type procyanidins for use in the present invention may be an oligomer composed of n monomeric, flavan-3-ol units of the formula: 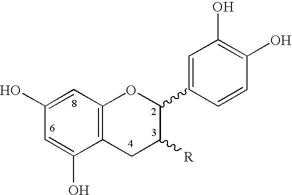
wherein
(i) the monomeric units are connected via interflavan linkages 4→6 and/or 4→8;
(ii) at least two of the monomeric units are additionally linked by an A-type interflavan linkage (4→8; 2→O→7) or (4→6; 2→O→7); and
(iii) n is 2 to 12.

It will be understood by a person of skill in the art that one of the two flavanol units linked by the A-type interflavanoid linkage must comprise two bonds at the 2- and 4-positions. Both of these have either α or β stereochemistry, i.e., the bonds are either 2α, 4α or 2β, 4β. These bonds connect to the 6- and 7-O-positions, or the 8- and 7-O-positions of the second flavanol unit linked by the A-type interflavan linkage. In constituent flavanol units of the oligomer which do not comprise A-type interflavan linkages at positions C-2 and C-4, the linkage at position C-4 can have either alpha or beta stereochemistry. The OH group at position C-3 of flavanol units has either alpha or beta stereochemistry. Flavan-3-ol (monomeric) units may be (+)-catechin, (−)-epicatechin and their respective epimers (e.g. (−)-catechin and (+)-epicatechin)).

An A-type procyanidin as defined above may be derivatized, for instance esterified, at one or more of the OH groups on one or more of the constituent flavan-3-ol units. A given flavan-3-ol unit may thus comprise one or more ester groups, preferably gallate ester groups, at one or more of the 3-, 5-, 7-, 3′- and 4′-ring positions. It may in particular be a mono-, di-, tri-, tetra- or penta-gallated unit.

Examples of the compounds useful for products, and in the methods, of the present invention include the compounds wherein the integer n is 3 to 12; 4 to 12; 5 to 12; 4 to 10; or 5 to 10. In some embodiments, n is 2 to 4, or 2 to 5, for example n is 2 or 3.

The A-type procyanidin may be epicatechin-(4β→8; 2β→O→7)-catechin (i.e., A1 dimer), or a pharmaceutically acceptable salt or derivative thereof, with the following formula:

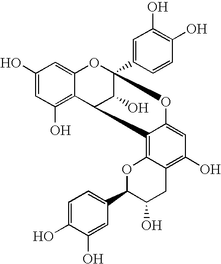

The A-type procyanidin may also be epicatechin-(4β→8; 2β→O→7)-epicatechin (i.e., A2 dimer) with the following formula:

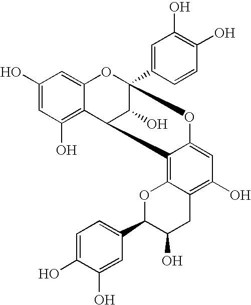

Alternatively, the A-type procyanidin may be an A-type trimer with the following formula:

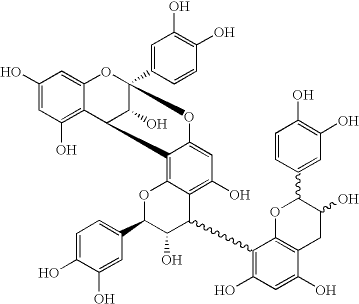

A-type procyanidins may be of natural origin or synthetically prepared. For example, A-type procyanidins may be isolated from peanut skins as described in Example 2, or as described in Lou et al., Phytochemistry, 51: 297-308 (1999), or Karchesy and Hemingway, J. Agric. Food Chem., 34:966-970 (1986), the relevant portions of each being hereby incorporated herein by reference. Mature red peanut skin contain about 17% by weight procyanidins, and among the dimeric procyanidins epicatechin-(4β→8; 2β→O→7)-catechin dominates, with smaller proportion of epicatechin-(4β→8; 2β→O→7)-epicatechin being present. However, in addition to procyanidins having (4→8; 2→O→7) double linkages, procyanidins having (4→6; 2→O→7) double linkages are also found in peanut skins.

Other sources of the above compounds are cranberries as described, for example in Foo et al., J. Nat. Prod., 63: 1225-1228, and in Prior et al., J. Agricultural Food Chem., 49(3):1270-76 (2001), the relevant portions of each being hereby incorporated herein by reference. Other sources include Ecdysanthera utilis (Lie-Chwen et al., J. Nat. Prod., 65:505-8 (2002)) and Aesculus hippocastanum (U.S. Pat. No. 4,863,956), the relevant portions of each being hereby incorporated herein by reference.

A-type compounds may also be obtained from B-type procyanidins via oxidation using 1,1-diphenyl-2-pycrylhydrazyl (DPPH) radicals under neutral conditions as described in Kondo et al., Tetrahedron Lett., 41: 485 (2000), the relevant portions of which are hereby incorporated herein by reference. Methods of obtaining natural and synthetic B-type procyanidins are well known in the art and are described, for example, in U.S. Pat. No. 6,670,390 to Romanczyk et al.; U.S. Pat. No. 6,207,842 to Romanczyk et al.; U.S. Pat. No. 6,420,572 to Romanczyk et al.; and U.S. Pat. No. 6,156,912 to Romanczyk et al.

The A-type procyanidins may be used in the compositions described herein and administered in the form of an extract (e.g. peanut skins extract) comprising A-type procyanidins as the main component.

The A-type procyanidins may be isolated and purified, i.e., they are separated from compounds with which they naturally occur (if the A-type procyanidin is of natural origin), or they are synthetically prepared, in either case such that the level of contaminating compounds (impurities) does not significantly contribute to, or detract from, the effectiveness of the A-type procyanidin. For example, an isolated and purified A1 dimer is separated from A2 dimer, with which it may occur in nature, to the extent achievable by the available commercially viable purification and separation techniques.

The other B-type procyanidins for use in the present invention may be of natural origin, for example, derived from a cocoa bean or another natural source, or prepared synthetically. For example, the B-type procyanidins and their derivatives are those described in U.S. Pat. No. 6,670,390 to Romanczyk et al., the relevant portions of which are hereby incorporated herein by reference. A person of skill in the art may select natural or synthetic procyanidins based on availability or cost. Procyanidins may be included in the composition in the form of a cocoa ingredient containing cocoa polyphenols, for example, chocolate liquor included in chocolate, or may be added independently of cocoa ingredients, for example, as an extract, extract fraction, isolated and purified individual compound, pooled extract fractions or a synthetically prepared compound.

The term “cocoa ingredient” refers to a cocoa solids-containing material derived from shell-free cocoa nibs such as chocolate liquor and partially or fully-defatted cocoa solids (e.g. cake or powder).

The B-type procyanidin oligomers may have from 2 to about 18, preferably from 2 to about 12, and most preferably from 2 to about 10 monomeric units. Alternatively, the oligomers may have from 3-18, preferably 3-12, and more preferably 3-10 monomeric units; or from 5-18, preferably 5-12 and more preferably 5-10 monomeric units. For example, oligomers may be dimers, trimers, tetramers, pentamers, hexamers, heptamers, octamers, nonamers and decamers. In the oligomer, monomers are connected via interflavan linkages of (4→6) and/or (4→8). Oligomers with exclusively (4→8) linkages are linear; while the presence of at least one (4→6) bond results in a branched oligomer. Also within the scope of the invention are oligomers comprising at least one non-natural linkage (6→6), (6→8), and (8→8). The synthesis of such non-naturally occurring oligomers is described in the International Appl. No. PCT/US/00/08234 published on Oct. 19, 2000 as WO 00/61547, the relevant portions of which are hereby incorporated herein by reference.

The B-type procyanidins may be prepared by extraction from cocoa beans, cocoa nibs, or cocoa ingredients such as chocolate liquor, partially defatted cocoa solids, and/or fully defatted cocoa solids. Preferably, the extract is prepared from a fully or partially defatted cocoa powder. Beans from any species of Theobroma, Herrania or inter- and intra-species crosses thereof may be used. The extract may be prepared from fermented, underfermented or unfermented beans, the fermented beans having the least amount of cocoa polyphenols and the unfermented the most. The selection of beans may be made based on the fermentation factor of the beans, for example, the extract may be made from the beans having a fermentation factor of about 275 or less. Optimizing the level of polyphenols in the cocoa ingredient and extract thereof by manipulating the degree of fermentation may be done as described in the International Appl. No. PCT/US97/15893 published as WO98/09533, the relevant portions of which are hereby incorporated herein by reference.

Cocoa polyphenols may be extracted from cocoa ingredients that have been processed using traditional methods of cocoa processing (described, for example, in Industrial Chocolate Manufacture and Use, ed. Beckett, S. T., Blackie Acad. & Professional, New York, 1997, such as in Chapters 1, 5 and 6) or using an improved processing method described in U.S. Pat. No. 6,015,913 to Kealey et al. that preserves polyphenols (by preventing their destruction) in cocoa ingredients in contrast to the traditional methods. The improved cocoa processing method omits the traditional roasting step. Thus, cocoa ingredients obtainable by (a) heating the cocoa bean for a time and a temperature sufficient to loosen the cocoa shell without roasting the cocoa nib; (b) winnowing the cocoa nib from the cocoa shell; (c) screw pressing the cocoa nib and (d) recovering the cocoa butter and partially defatted cocoa solids which contain preserved levels of cocoa polyphenols, may be used. The method retains a much higher level of higher procyanidin oligomers than traditional processing methods. Cocoa solids produced by this method may contain greater than 20,000 μg of total flavanol and/or procyanidins per gram nonfat solids; preferably greater than 25,000 μg, more preferably greater than 28,000 μg, and most preferably greater than 30,000 μg. For purposes of this invention, the total flavanol and/or procyanidin amounts may be determined as described in Example 4.

B-type procyanidins may be extracted from the sources indicated above, or any other polyphenol or flavanol or procyanidin containing source, using solvents in which the polyphenols dissolve. Suitable solvents include water or organic solvent such as methanol, ethanol, acetone, isopropyl alcohol and ethyl acetate. Solvent mixtures may also be used. When water is used as the solvent, it may be slightly acidified, for example with acetic acid. Examples of some solvents are mixtures of water and organic solvent, for example aqueous methanol, ethanol or acetone. Aqueous organic solvents may contain, for example, from about 50% to about 95% of organic solvent. Thus, about 50%, about 60%, about 70%, about 80% and about 90% organic solvent in water may be used. The solvent may also contain a small amount of acid such as acetic acid, for example, in the amount of about 0.5% to about 1.0%. The composition of the extracts, i.e., the representation (i.e., oligomeric profile) and the amount of procyanidin oligomers, will depend on the choice of solvents. For example, the water extract contains primarily monomers, the ethyl acetate extract contains monomers and lower oligomers, mainly dimers and trimers, and the aqueous methanol, ethanol or acetone extract contains monomers and a range of higher oligomers. One of the solvents for extraction of monomer as well as higher procyanidin oligomers is about 70% acetone. However, any extract containing polyphenols is useful in the invention. The methods of cocoa polyphenol extraction are known in the art and are described, for example, in the U.S. Pat. No. 5,554,645 to Romanczyk et al. and the International Appl. No. PCT/US97/05693, published as WO97/36497. Thus, in one embodiment, the cocoa extract is prepared by reducing cocoa beans to cocoa powder, defatting the powder, extracting the cocoa polyphenols, and purifying the extract. The cocoa powder can be prepared by freeze-drying the cocoa beans and pulp, depulping and dehulling the freeze-dried cocoa beans, and grinding the dehulled beans.

The B-type cocoa polyphenol extract may be purified, for example, by removal of the caffeine and/or theobromine, and further purified by gel permeation chromatography and/or High Pressure Liquid Chromatography (HPLC). Gel permeation chromatography (e.g. on Sephadex LH-20) may be used to enrich the extract for higher procyanidin oligomers. For example, the eluate containing monomers and lower oligomers may not be collected until the oligomer(s) of choice begins eluting from the column. An example of such an extract is known in the art and is described in Example 5 of the International Appl. No. PCT/US97/05693, published as WO97/36497, the relevant portions of which are hereby incorporated by reference herein. By using preparative HPLC, for example, normal phase HPLC, the extract may be fractionated, for example, into monomeric and oligomeric fractions containing at least 50% by weight of the monomer or specific oligomer(s). When a particular fraction contains the monomers or any of the lower oligomers (e.g. dimers, trimers or tetramers fraction), the fraction contain about 90 to 95% by weight of the particular oligomeric fraction. The desired fractions may be pooled after separation to obtain a combination of oligomers of choice for example to contain oligomers 3-10 or 5-10. A person of skill in the art can manipulate the chromatographic conditions to achieve the desired procyanidin profile in view of the guidance in this specification, general knowledge in the art and, for example, the teachings of U.S. Pat. No. 5,554,645 to Romanczyk et al. and the International Appl. No. PCT/US97/05693, published as WO97/36497.

The monomeric fraction typically contains a mixture of monomers epicatechin and catechin; and the oligomeric fraction typically contains a mixture of dimers (in a dimer fraction), trimers (in a trimer fraction), tetramers (in a tetramer fraction), etc. Mixtures of monomers and oligomers occur in isolated fractions because cocoa contains more than one type of each of monomer, dimer, etc. The oligomeric variability occurs as a result of two monomers, epicatechin and catechin, that are building blocks of procyanidins, as well as the chemical bond connecting monomers in the oligomer. Thus, cocoa dimers are primarily B2 and B5, each of which contains two monomers of epicatechin. Individual monomers and oligomers may be obtained using reversed-phase HPLC, e.g. using a C18 column.

B-type cocoa polyphenol may be used in the compositions of the invention as a cocoa extract, e.g. solvent-derived extract, cocoa fraction, isolated compounds or in the form of a cocoa ingredient or a chocolate containing an effective amount of cocoa flavanols and/or procyanidins. The cocoa ingredients may be prepared using traditional cocoa processing procedures but is preferably prepared using the method described in U.S. Pat. No. 6,015,913 to Kealey et al. Alternatively, to enhance the level of cocoa polyphenols, chocolate liquor and cocoa solids prepared from cocoa beans having a fermentation factor of about 275 or less may be used. These ingredients have cocoa polyphenol content that is higher than can be obtained using traditional cocoa processing methods (e.g. with roasting) and fully fermented beans. The chocolate may be prepared using conventional techniques from the ingredients described above or using an improved process for preserving cocoa polyphenols during chocolate manufacturing as described in the International Appl. No. PCT/US99/05414 published as WO99/45788, the relevant portions of which are hereby incorporated herein by reference. A chocolate prepared by at least one of the following non-traditional processes is referred to herein as a “chocolate having a conserved amount of cocoa polyphenols”: (i) preparing cocoa ingredients from underfermented or unfermented cocoa beans; (ii) preserving cocoa polyphenol during cocoa ingredient manufacturing process; and (iii) preserving cocoa polyphenol during chocolate manufacturing process.

Synthetic B-type procyanidins may also be used and are prepared by methods known in the art and as described for example in the International Appl. No. PCT/US98/21392 published as WO99/19319, the relevant portions of which are hereby incorporated herein by reference.

Flavanol and/or procyanidin derivatives may also be useful. These include esters of monomer and oligomers such as the gallate esters (e.g. epicatechin gallate and catechin gallate); compounds derivatized with a saccharide moiety such as mono- or di-saccharide moiety (e.g. β-D-glucose), glycosylated monomers and oligomers, and mixtures thereof; metabolites of the procyanidin monomers and oligomers, such as the sulphated, glucouronidated, and methylated forms except for the enzyme cleavage products of procyanidins generated by colonic microflora metabolism. The derivatives may be from natural sources or prepared synthetically.

The foods comprising the dimer and dimer digallates of the invention and/or A-type and/or other B-type procyanidins and optionally another vascular-protective/treatment agent may be adapted for human or veterinary use, and include pet foods. The food may be other than a confectionery, however, the preferred food is a confectionery such as a standard of identity (SOI) and non-SOI chocolate, such as milk, sweet and semi-sweet chocolate including dark chocolate, low fat chocolate and a candy which may be a chocolate covered candy. Other examples include a baked product (e.g. brownie, baked snack, cookie, biscuit) a condiment, a granola bar, a toffee chew, a meal replacement bar, a spread, a syrup, a powder beverage mix, a cocoa or a chocolate flavored beverage, a pudding, a rice cake, a rice mix, a savory sauce and the like. If desired, the foods may be chocolate or cocoa flavored. Food products may be chocolates and candy bars, such as granola bars, containing nuts, for example, peanuts, walnuts, almonds, and hazelnuts. It should be noted that the addition of nuts with skins to the food described herein may also increase the total polyphenol content since, for example, peanut skins contain about 17% flavanols and procyanidins and almond skins contain about 30% flavanols and procyanidins. Ground peanut skins may be added to the compositions of the invention. In one embodiment, the nut skins, e.g. peanut skins, are added to the nougat of a chocolate candy.

In certain embodiments, the non-chocolate food product contains from about at least 5 micrograms/g to about 10 mg/g, and, for example, at least 5 micrograms/g food product, preferably at least 10 microgram/g, more preferably at least 100 micrograms/g of dimer digallates and/or flavanols and/or B-type procyanidins and/or A-type procyanidins. If desired, the non-chocolate food products can contain much higher levels of cocoa procyanidins than those found in the chocolate food products described below.

The chocolate confectionery may be milk or dark chocolate. In certain embodiments, the chocolate comprises at least 3,600 micrograms, preferably at least 4,000 micrograms, preferably at least 4,500 micrograms, more preferably at least 5,000 micrograms, and most preferably at least 5,500 micrograms of dimer digallates and/or flavanols and/or B-type procyanidins and/or A-type procyanidins each per gram of chocolate, based on the total amount of nonfat cocoa solids in the product. In other embodiments, the chocolate contains at least 6,000 micrograms, preferably at least 6,500 micrograms, more preferably at least 7,000 micrograms, and most preferably at least 8,000 micrograms of dimer digallates and/or flavanols and/or B-type procyanidins and/or A-type procyanidins per gram, and even more preferably 10,000 micrograms/g based on the nonfat cocoa solids in the product.

A milk chocolate confectionery may have at least 1,000 micrograms, preferably at least 1,250 micrograms, more preferably at least 1,500 micrograms, and most preferably at least 2,000 micrograms dimer digallates and/or flavanols and/or B-type procyanidins and/or A-type procyanidins each per gram of milk chocolate, based on the total amount of nonfat cocoa solids in the milk chocolate product. In the preferred embodiment, the milk chocolate contains at least 2,500 micrograms, preferably at least 3,000 micrograms, more preferably at least 4,000 micrograms, and most preferably at least 5,000 micrograms dimer digallates and/or flavanols and/or B-type procyanidins and/or A-type procyanidins each per gram of milk chocolate, based on the total amount of nonfat cocoa solids in the milk chocolate product.

L-arginine may be added to food products in the amount that can vary. Typically, cocoa contains between 1 to 1.1 grams of L-arginine per 100 grams of partially defatted cocoa solids. It can range from 0.8 to 1.5 per 100 grams of cocoa. In some embodiments, the chocolate food products of this invention contain L-arginine in an amount greater than that which naturally occurs in the cocoa ingredients. Knowing the amount of cocoa ingredients and L-arginine used in the food product, one of ordinary skill in the art can readily determine the total amount of L-arginine in the final product. The food product will generally contain at least 5 micrograms/g, preferably at least 30 micrograms/g, or at least 60 micrograms/g, even more preferably at least 200 micrograms/g food product.

A daily effective amount of dimer digallates and/or flavanols and/or A-type and/or B-type procyanidins may be provided in a single serving. Thus, a confectionery (e.g. chocolate) may contain at least about 100 mg/serving (e.g. 150-200, 200-400 mg/serving).

The invention is further described in the following non-limiting examples.

### EXAMPLES

### Example 1

Synthesis of Dimer Digallates

Dimer digallates may be synthesized as described in U.S. Pat. No. 6,420,572, hereby incorporated herein by reference, from which this example is being reproduced.

### A. Preparation of (2R,3S, trans)-5,7,3,′4′-Tetra-O-benzylcatechin

A solution of (+)-catechin (65.8 g, 226.7 mmol, anhydrous), dissolved in anhydrous dimethylformamide (DMF, 720 mL), was added dropwise, at room temperature over a period of 80 min, to a stirred suspension of sodium hydride, 60% in oil, (39 g, 975 mmol, 4.3 eq.) in DMF (180 mL). (S. Miura, et al., Radioisotopes, 32, 225-230(1983)). After stirring for 50 min, the flask was placed in a −10° C. NaCl/ice bath. Benzyl bromide (121 mL, 1.02 mol, 4.5 eq.) was added dropwise within 80 min. and the brown reaction mixture warmed to room temperature, with stirring, overnight. The resulting reaction mixture was evaporated and the resulting candy-like solid was dissolved, with heating and stirring, in two portions of solvent each consisting of 200 mL of chloroform (CHCl₃) and 100 mL of water. The phases were separated, the aqueous phase extracted with CHCl₃ (20 mL), and the combined organic phases washed with water (100 mL), dried over magnesium sulfate (MgSO₄) and evaporated. The residue was purified by chromatography on silica gel (42×10 cm; ethyl acetate/chloroform/hexane 1:12:7) to provide, after evaporation and drying in vacuo, 85 g crude product, which was recrystallized from trichloroethylene (1.3 L) to provide 35.1 g (24%) of an off-white powder. ¹H NMR (CDCl₃) δ7.47-7.25 (m, 20 H), 7.03 (s, 1 H), 6.95 (s, 2 H), 6.27, 6.21 (ABq, 2 H, J=2 Hz), 5.18 (s, 2 H), 5.17 (narrow ABq, 2 H), 5.03 (s, 2 H), 4.99 (s, 2 H), 4.63 (d, 1 H, J=8.5 Hz), 4.00 (m, 1 H), 3.11, 2.65 (ABq, 2 H, J=16.5 Hz, both parts d with J=5.5 and 9 Hz, resp.), 1.59 (d, 1 H, J=3.5 Hz); IR (film) 3440 (br), 1618, 1593, 1513, 1499, 1144, 1116, 733, 696 cm⁻¹; MS m/z 650 (M+, 0.5%),319, 181, 91.

Alternatively, the tetra-O-benzyl (+)-catechin may be prepared using the method described by H. Kawamoto et al., Mokazai Gakkaishi, 37, (5) 488-493 (1991), using potassium carbonate and benzyl bromide in DMF. Partial racemization of catechin, at both the 2- and 3-positions, was observed by M.-C. Pierre et al., Tetrahedron Letters, 38, (32) 5639-5642 (1997).

### B. Preparation of (2R)-5,7,3′,4′ Tetrakis(benzyloxy)flavan-3-one

Freshly prepared Dess-Martin periodinane (39.0 g, 92 mmol, prepared by the method of D. B. Dess and J. C. Martin, J. Am. Chem. Soc. 113, 7277-7287 (1991) and R. E. Ireland and L. Liu, J. Org. Chem. 58, 2899 (1993)), was added at room temperature, all at once, to a stirred solution of the tetra-O-benzylcatechin according to Part A (54.4 g, 83.8 mmol) in methylene chloride (420 mL). Within 1.5 h, approx. 30 mL of water-saturated methylene chloride was added dropwise to the reaction mixture to form a turbid amber-colored solution. (S. D. Meyer and S. L. Schreiber, J. Org. Chem., 59, 7549-7552 (1994)) Twenty minutes thereafter, the reaction mixture was diluted with a saturated solution of sodium carbonate (NaHCO.sub.3, 500 mL) and a 10% aqueous solution of Na₂S₂O₃.5H₂O (200 mL). The phases were separated and the aqueous phase extracted with 50 mL of methylene chloride. The combined organic phases were filtered over silica gel (24×9 cm, chloroform/ethyl acetate 9:1). The eluate was evaporated and dried in vacuo to obtain 50.1 g (92%) of the ketone, which was purified by recrystallization from chloroform/ether: mp 144-144.5° C.; [α]_{D}+38.5°, [α]₅₄₆+48.7° (chloroform, c 20.8 g/L); ¹H NMR (CDCl₃) δ 7.45-7.26 (m, 20 H), 6.96 (s, 1 H), 6.88, 6.86 (ABq, 2 H, J=8 Hz, B part d with J=1.5 Hz), 6.35 (narrow ABq, 2 H), 5.24 (s, 1 H), 5.14 (s, 2 H), 5.10 (narrow ABq, 2 H), 5.02 (s, 2 H), 5.01 (s, 2 H), 3.61, 3.45 (ABq, 2 H, J=21.5 Hz).

### C. Preparation of 5,7,3′,4′-Tetra-O-benzylepicatechin

A 1 M solution of lithium tri-sec-butylborohydride in tetrahydrofuran, herein after THF, (100 mL, L-Selectride®, sold by the Aldrich Chemical Co, Inc., Milwaukee, Wis.) was added, under an argon atmosphere, to a stirred, 0° C. solution of anhydrous lithium bromide, LiBr, (34.9 g, 402 mmol) in 100 mL anhydrous THF. The resulting mixture was cooled to −78° C., using an acetone/CO₂ bath, followed by dropwise addition of a solution of the flavanone according to Part B (50.1 g, 77.2 mmol) in 400 mL of anhydrous THF, over a period of 50 min. Stirring was continued at −78° C. for 135 min. The cooling bath was removed and 360 mL of 2.5 M aqueous sodium hydroxide (NaOH) was added to the reaction mixture. The reaction flask was placed in a room temperature water bath and a mixture of 35% aqueous H₂O₂ (90 mL) and ethanol (270 mL) was added over a period of 130 min. Stirring was continued overnight. Chloroform (700 mL) was added to dissolve the crystallized product, the phases were separated, the aqueous phase was extracted with CHCl₃ (50 mL), the combined organic phases were dried over MgSO₄, evaporated and dried in vacuo to provide 56.6 g of crude product. This material was dissolved in 600 mL of a boiling mixture of ethyl acetate (EtOAc) and ethanol (EtOH) (2:3), and allowed to crystallize at room temperature, then in the refrigerator. The product was isolated by suction filtration, washed with 2×50 mL of cold (−20° C.) EtOAc/EtOH (1:3), and dried in vacuo first at room temperature, then at 80° C. to obtain 35.4 g (70%) of a light yellow solid. The evaporated mother liquor was filtered over silica gel, SiO₂, (14×6.5 cm, CHCl₃, then CHCl₃/EtOAc 12:1), the eluate concentrated to 40 mL, and the residue diluted with 60 mL of ethanol, to obtain an additional 5.5 g (11%) of the O-benzylepicatechin as a yellowish solid: mp 129.5-130° C. (from EtOAc/EtOH); [α]_{D}−27.7° [α]₅₄₆−33.4° (EtOAc, c 21.6 g/L); ¹H NMR (CDCl₃) δ 7.48-7.25 (m, 20 H), 7.14 (s, 1 H), 7.00, 6.97 (ABq, 2 H. J=8.5 Hz, A part d with J=1.5 Hz), 6.27 (s, 2 H), 5.19 (s, 2 H), 5.18 (s, 2 H), 5.02 (s, 2 H), 5.01 (s, 2 H), 4.91 (s, 1 H), 4.21 (br s, 1 H), 3.00, 2.92 (ABq, 2 H, J=17.5 Hz, both parts d with J=1.5 and 4 Hz, resp.), 1.66 (d, 1 H, J=5.5 Hz); Anal. Calcd. for C₄₃H₃₈O₆: C, 79.36; H, 5.89. Found: C, 79.12: H, 5.99.

### D. Preparation of (2R,3S,4S)-5,7,3′,4′-Tetra O-benzyl-4-(2-hydroxyethoxy)epicatechin

Ethylene glycol (6.4 mL, 115 mmol, 5.8 eq.) was added, at room temperature, with stirring, to a solution of the tetra-O-benzylepicatechin according to Part C (12.75 g, 19.6 mmol) in 130 mL of anhydrous methylene chloride, followed by addition of 2,3-dichloro-5,6-dicyano-1,4-benzoquinone (DDQ, 8.9 g, 39.2 mmol, 2.0 eq.), at one time, with vigorous stirring. (J. A. Steenkamp, et al., Tetrahedron Letters, 26, (25) 3045-3048 (1985)). After approximately 2 hours, 4-dimethylaminopyridine (DMAP, 4.8 g, 39.2 mmol) was added to the reaction mixture, resulting in the formation of a dark green precipitate. After stirring for an additional 5 minutes, 100 g of silica gel was added, and the mixture was concentrated under reduced pressure. The residue was placed on top of a silica gel column (11×6.5 cm) which was eluted with EtOAc/hexane (1:1), and the eluate was concentrated under reduced pressure. The resulting crude material was re-purified by chromatography on silica gel (39×10 cm, EtOAc/hexane (1:2), followed by EtOAc/hexane (2:3)) to provide, after evaporation and drying, in vacuo, 7.3 g (52%) of the benzyl-4-(2-hydroxyethoxy) epicatechin, as a foam or solid, which was recrystallized from acetonitrile: mp 120-121° C.; 1H NMR (CDCl₃) δ 7.48-7.26 (m, 20 H), 7.14 (d, J=1.5 Hz), 7.02, 6.97 (ABq, 2 H, J=8 Hz, A part d with J=1.5 Hz), 6.29, 6.26 (ABq, 2 H, J=2 Hz), 5.19 (s, 2 H), 5.17 (s, 2 H), 5.10 (s, 1 H), 5.08, 5.02 (ABq, 2 H, partially concealed), 5.00 (s, 2 H), 4.59 (d, 1 H, J=2.5 Hz), 3.95 (br, 1 H), 3.82-3.74 (m, 1 H), 3.72-3.57 (m, 3 H), 2.17 (br, 1 H), 1.64 (d, 1 H, J=5.5 Hz); IR (film) 3450 (br), 1616, 1592, 1512, 1152, 1114, 735, 697 cm.sup.−1. Anal. Calcd. for C₄₅H₄₂O₈: C, 76.04; H, 5.96. Found: C, 76.57; H, 6.02.

### E. Preparation of O-Benzylepicatechin (4β→8) Oligomers

To a cold (0° C.), stirred solution of the benzyl-4-(2-hydroxyethoxy) epicatechin according to Part D (3.28 g, 4.6 mmol) and the tetra-O-benzyl-epicatechin according to Part C (12.0 g, 18.4 mmol, 4 eq.) in anhydrous THF (40 mL) and anhydrous methylene chloride (50 mL), was added dropwise, in 10 min, titanium tetrachloride (4.6 mL of 1 M TiCl₄ in methylene chloride). (H. Kawamoto et al., Mokazai Gakkaishi, 37, (5) 448-493 (1991)) The resulting amber-colored 35 solution was stirred in the ice bath for 5 min, then at room temperature for 90 min. The reaction was terminated by addition of 30 mL of saturated aqueous NaHCO₃ and 100 mL of water (resulting pH: 8). The resulting mixture was extracted with methylene chloride (2×20 mL). The combined organic layers were washed with 50 mL of water, dried over MgSO₄, evaporated and dried in vacuo. The resulting glass deposited a pink solid upon dissolution in methylene chloride (CH₂Cl₂) and standing at room temperature. The solid was filtered off, washed with 3×15 mL of CH₂Cl₂/hexane (1:1), and dried in vacuo to obtain 6.1 g of recovered tetra-O-benzylepicatechin. From the evaporated mother liquor, the oligomers were isolated by column chromatography on silica gel (45×5.2 cm). Elution with CH₂Cl₂/hexane/EtOAc (13:13:1) provided an additional 4.9 g of recovered tetra-O-benzylepicatechin, followed by 2.17 g of crude O-benzyl dimer. Elution of the dimer was completed using methylene chloride/hexane/EtOAc (10:10:1). Elution of 0.98 g of crude O-benzyl trimer and 0.59 g of higher oligomers was obtained using methylene chloride/hexane/EtOAc (8:8:1 to 6:6:1). The dimer and the trimer were further purified by preparative HPLC on a silica gel column, using ethyl acetate/hexane or ethyl acetate/isooctane as eluent. Peak detection was performed with a UV detector at 265 or 280 nm. Trimer: MS (MALDI-TOF, DHBA matrix) m/z (M+H⁺) 1949.4; calcd. for C₁₂₉H₁₁₀O₁₈: 1947.8; (M+Na⁺) 1971.2; calcd. for C₁₂₉H₁₁₀O₁₈Na: 1969.8; (M+K⁺) 1988.3; calcd. for C₁₂₉H₁₁₀O₁₈K: 1985.7.

### F. Preparation of Epicatechin Dimer

To a solution of the O-benzyl-dimer according to Part E (22.3 mg, 17.2.μmol) in 0.5 mL of ethyl acetate was added sequentially, 2 mL of methanol and 7.2 mg of 10% Pd/C. The mixture was stirred under 1 bar of H₂ for 3 hours and filtered over cotton. The filtration residue was washed with methanol and the combined filtrates were evaporated. An NMR spectrum of the crude product indicated the presence of benzylated material. The procedure was therefore repeated, with the amount of catalyst increased to 17.5 mg and the time extended to 3.7 h. The crude polyphenol dimer (9.6 mg) was purified by preparative HPLC (C₁₈ reverse phase column water/methanol (85:15) with addition of 0.5% acetic acid, detection at 265 nm) to provide 4.5 mg (45%) of polyphenol dimer as an amorphous film. ¹H NMR (300 MHz, acetone-d.₆/D₂O 3:1 (v/v), TMS) δ 7.19 (br, 1 H), 7.01 (overlapping s+br, 2 H), 6.86-6.65 (m, 4 H), 6.03 (br, 3 H), 5.10 (br, 1 H), 5.00 (br, 1 H), 4.69 (br, 1 H), 3.97 (s, 1 H), 2.92, 2.76 (br ABq, 2 H, J=17 Hz); MS (MALDI-TOF, DHBA matrix) m/z (M+K⁺) 616.8; calcd. for C₃₀H₂₆O₁₂K: 617.1; (M+Na⁺) 600.8; calcd. for C₃₀H₂₆O₁₂Na: 601.1.

### G. Preparation of O-Benzylepicatechin Dimer Bisgallate

To a solution of tri-O-benzyl gallic acid (38 mg, 87 μmol, 5 eq.), DMF (1 μL) in methylene chloride (0.6 mL), was added oxalyl chloride (15 μL, 172 μmol, 10 eq.). The resulting reaction mixture was stirred at room temperature for approximately 1 hour, evaporated and dried in vacuo to provide tri-O-benzyl galloyl chloride. A solution of the O-benzyl-dimer according to Part E (22.5 mg, 17.3 μmol) in anhydrous pyridine (0.5 mL) was added to the crude galloyl chloride at room temperature, and the resulting mixture was stirred for 44.5 h. After addition of 20 μL of water, stirring was continued for 2.5 h, followed by addition of 10 mL of 5% HCl. The resulting mixture was extracted with methylene chloride (3×5 mL), the combined organic phases were dried over MgSO₄, evaporated and purified by filtration over silica gel using with EtOAc/CHCl₃ (1:19). Concentration of the eluate and drying in vacuo yielded 36.0 mg (97%) of the O-benzyl dimer bisgallate as a colorless film: [α]_{D}−53.3°, [α]₅₄₆−65.6° (CH₂Cl₂, c 15.7 g/L); IR (film) 1720, 1591, 1498, 1428, 1196, 1112, 736, 696 cm⁻¹; MS (MALDI-TOF, DHBA matrix) m/z (M+K.⁺) 2181.8; calcd. for C₁₄₂H₁₁₈O₂₀K: 2181.8; (M+Na.⁺) 2165.9; calcd. for C₁₄₂H₁₁₈O₂₀ Na: 2165.8.

### H. Preparation of Epicatechin Dimer Bisgallate

To a solution of the O-benzyl dimer bisgallate according to Part G (33.8 mg, 15.8 μmol) in 4 mL of THF was added sequentially 4 mL of methanol, 0.2 mL of water, and 42 mg of 20% Pd(OH)₂/C. The mixture was stirred under 1 bar of H₂ for 75 minutes and filtered over cotton. The filtration residue was washed with 2.2 mL of methanol/H₂O (10:1) and the combined filtrate was concentrated under reduced pressure to provide 14.2 mg of yellowish, amorphous crude product. A 7.2 mg aliquot was purified by preparative HPLC (silica gel, ethyl acetate/hexane; detection at 280 nm) to yield 5.0 mg (71%) of the polyphenol dimer bisgallate as a turbid pinkish glass from which small amounts of ethanol and acetic acid could not be removed: ¹H NMR (acetone-d₆/D₂O 3:1 v/v, TMS, most signals broad) δ 7.08 (s, 2 H, sharp), 7.1-6.7 (m, 7 H), 6.66 (d, 1 H, sharp, J=8 Hz), 6.17 (s, 1 H), 5.94 (s, 2 H), 5.70 (s, 1 H), 5.49 (s, 1 H), 5.44 (s, 1 H), 4.9 (very br, 1 H), 4.80 (s, 1 H), 3.08, 2.88 (ABq, 2 H, J=17 Hz, A part d, J=4 Hz); MS (MALDI-TOF, DHBA matrix) m/z (M+Na.⁺) 904.9; calcd. for C₄₄H₃₄O₂₀Na: 905.2.

### Example 2

Extraction and Isolation of A-type Procyanidins

Extraction

Finely ground peanut skins (498 g) were defatted with hexane (2×2000 mL). Hexane was removed by centrifugation at ambient temperature, 5 min at 3500 rpm, and discarded. Residual hexane was allowed to evaporate overnight. The following day, defatted peanut skins were extracted for 2 hours at ambient temperature with acetone:water:acetic acid (70:29.5:0.5 v/v/v) (2×2000 mL). Extracts were recovered by centrifugation (ambient temperature, 5 min at 3500 rpm). Organic solvents were removed by rotary evaporation under partial pressure (40° C.). Aqueous portion of extraction solvent was removed by freeze drying to provide a brown-red crusty solid (51.36 g).

Gel Permeation of Crude Peanut Skin Extract

Crude peanut skin extract (24 g), obtained as described above, was dissolved in 70% methanol (150 mL), refrigerated for 1 hour, vortexed for 3 sec, then centrifuged at ambient temperature, for 5 min at 3500 rpm. The supernatant was loaded atop a large column containing Sephadex LH-20 (400 g) preswollen in methanol. Column was eluted isocratically with 100% methanol at a flow rate of 10 mL/min. Twenty nine fractions, 250 mL each, were collected and combined in accordance to their composition as determined by NP-HPLC (Adamson et al., J. Ag. Food Chem., 47: 4184-4188, 1999) to give a total of eight fractions (i-viii). Fraction i contained monomers epicatechin and catechin, fraction ii-vii contained dimers, trimers or mixtures thereof. Fraction v (1.8 g) and vii (2.7 g) contained a preponderance of dimers and trimers, respectively, and were selected for further purification.

Purification of A-Type Dimers and Trimers

Fraction v (1.8 g) was dissolved in 0.1% acetic acid in 20% methanol (40 mg/mL). Injection volumes were 2 mL. Separations were conducted on a Hypersil ODS (250×23 mm) under gradient conditions. Mobile phases consisted of 0.1% acetic acid in water (mobile phase A) and 0.1% acetic acid in methanol (mobile phase B). Gradient conditions were: 0-10 min, 20% B isocratic; 10-60 min, 20-40% B linear; 60-65 min, 40-100% B linear. Separations were monitored at 280 nm. Fractions with equal retention times from several preparative separations were combined, rotary evaporated at 40° C. under partial vacuum and freeze dried. Five fractions (a-e) were obtained. Fractions d and e were characterized by LCMS as dimers A1 and A2, respectively. In addition to A1 and A2 dimers, four different dimers were previously isolated from peanut skins (Lou et al., Phytochemistry 51, 297-308, 1999).

Fraction vii was purified as described above to obtain a single trimer with an A-linkage having the formula represented above.

The structures of purified compounds were confirmed by Mass Spectroscopy, and the purity of the compounds was determined using HPLC at UV 280 nm. A1 dimer was 95% pure, A2 dimer was 91% pure, and A trimer was 84% pure.

### Example 3

Effect of Dimer and Dimer Digallates on NO Production and Vasorelaxation

Several compounds were investigated for their effect on nitric oxide (NO) production and vasorelaxation using serum-free human umbilical vein endothelial cell (HUVEC) culture system in vitro and rabbit aortic ring model ex vivo, respectively. NO production by endothelial cells and relaxation of pre-constricted aortic rings are two main markers for evaluating vascular effects of test compounds.

In vitro Experiments

HUVECs obtained from a single donor were cultured in serum free, low protein (0.5 g/l), antibiotic-free cell culture medium supplemented with essential growth factors, nutrients and minerals. The cultured cell expressed endothelial markers (von Willebrand factor, CD31 antigen, uptake of Dil-Ac-LDL) and exhibited the typical “cobble-stone morphology” when grown to confluence. The cell culture medium was substituted with apo-transferrin, superoxide dismutase, and catalase to exclude secondary effects of test compounds involving their auto-oxidation mediated hydrogen peroxide formation.

NO production was evaluated by measuring the total amount of all major nitric oxide end products (NOx, including nitrate, nitrite, nitrosothiols) present in the cell culture medium. For this purpose NOx were directly reduced by vanadium(III)chloride/HCl at 95° C. yielding NO. The amount of NO released from the culture medium was subsequently evaluated by measuring the chemiluminescence emitted during the stoichiometrical reaction between ozone and NO using a NO Analyzer (Sievers Instruments, Inc. Boulder, Colo.).

### Experiment 1

Test compounds were evaluated with respect to their potential to acutely (2 hour) and chronically (5 doses given in 24 hour intervals) modulate NO production. Positive controls (acetylcholine and/or histamine) and negative control L-NNMA (NO synthase inhibitor) were included in all experiments. Cell counts and total protein were used to assess intra-assay variation. Potential toxic effects of tested compounds were also monitored (MTT reduction was measured).

The data presented herein were obtained from three experiments and were expressed as the concentration of NO present (in μmol/l) (as NOx) in the cell culture medium+/−standard deviation (SD). The data were corrected for the NOx intrinsically present in the fully supplemented cell culture medium and normalized with respect to the volume of media from which the sample was drawn. Data were analyzed using Student's t-test with a 95% level of confidence. P values equal to or less than 0.05 were defined as statistically significant.

The following compounds were tested in vitro:
C-(4α→8)-C
EC-(4β→8)-EC
EC-(4β→8)-C
   Methylated B2 dimers (mixture of dimers obtained by extraction from cocoa and subsequently methylated)
EC-(4β→8)-EC digallate
EC-(4β→8)(OMethyl)4-EC digallate

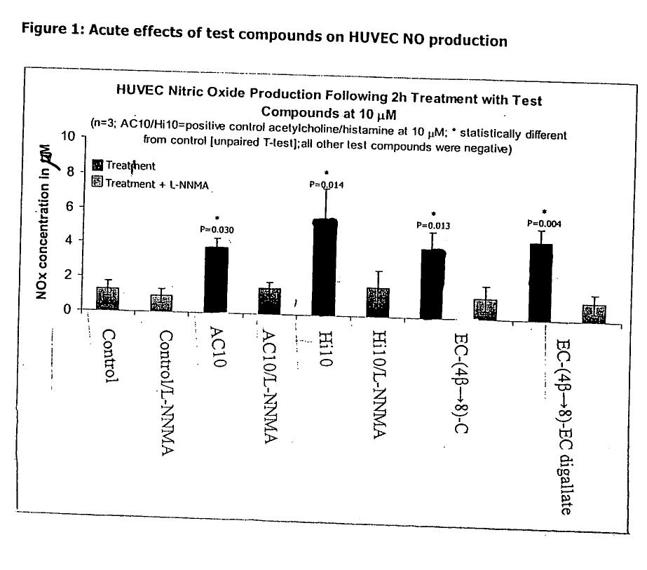

For the acute effect test, HUVECs were incubated with the test compounds for 2 hours at concentrations of 100 nM, 1 μM, and 10 μM at 37° C. and 5% CO₂. The positive control, acetylcholine and histamine, mediated a statistically significant increase in NO production as compared to vehicle, and L-NNMA completely attenuated the acetylcholine and histamine-NO production. (FIG. 1)

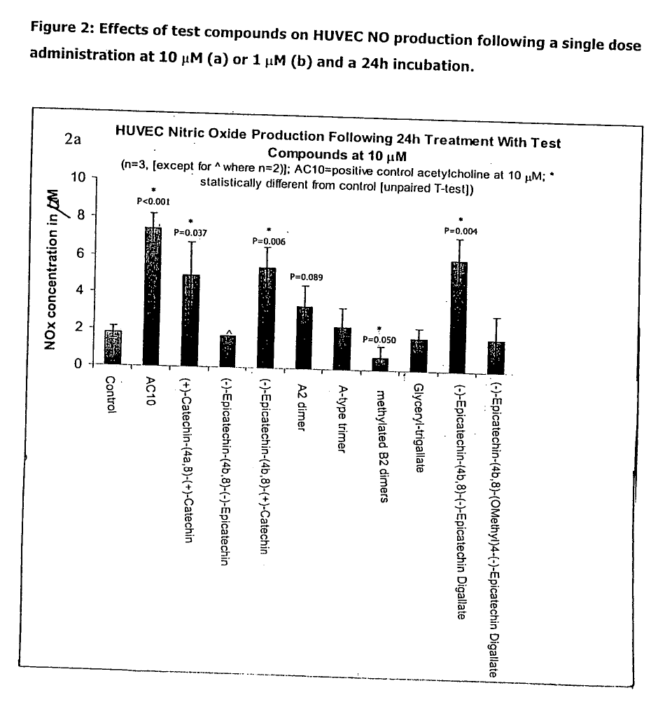

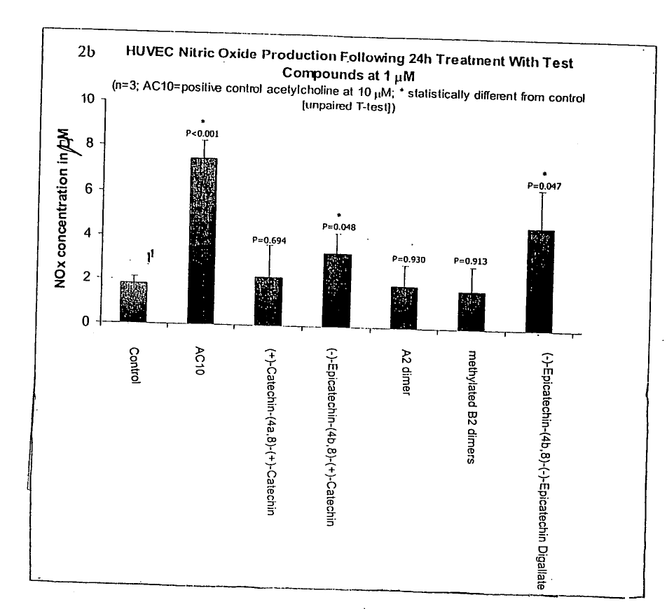

EC-(4β→8)-C and EC-(4β→8)-EC digallate demonstrated a statically significant ability to modulate NO production when administered at a concentration of both 10 μM (FIG. 1). However, EC-(4β→8)-C was later discovered to contain significant contamination with A1 dimer; and when re-purified, it no longer exhibited the NO modulating effect. Other compounds failed to significantly modulate NO synthesis following 2 h of incubation. After 24 h of a single dose administration, C-(4α→8)-C, EC-(4β→8)-C and EC-(4β→8)-EC digallate caused increased NO production when used at a concentration of 10 μM, and EC-(4β→8)-C and EC-(4β→8)-EC digallate when administered at a concentration of 1 mM. Methylated B2 dimers mediated a significant decrease of NO production as compared to control when administered at a concentration of 10 μM (FIGS. 2a and 2b). Test compounds showed no significant effects at a concentration of 100 nM. Based on the MTT assay, the test compounds did not have toxic effects.

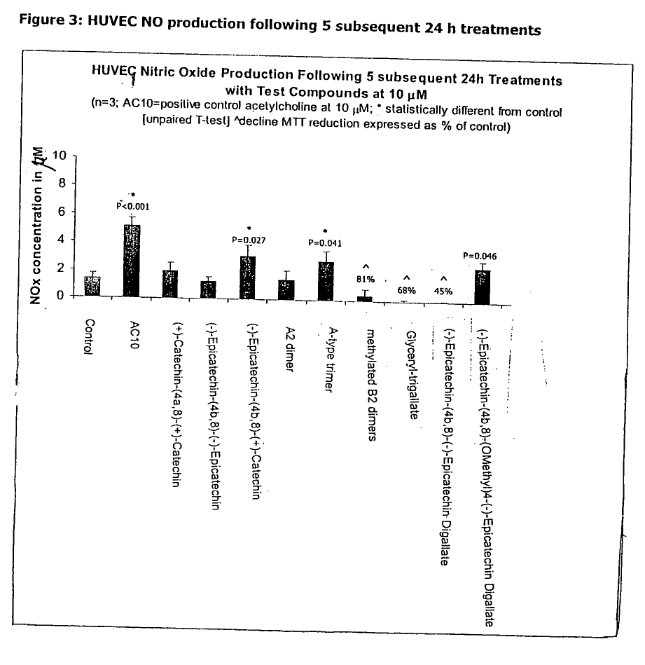

For the chronic effect test, HUVECs were incubated with 5 subsequent doses of test compounds, each for 24 hours. After each 24 hour treatment, culture medium was replaced. C-(4α→8)-C did not modulate NO production as it had with single does treatment. Methylated B2 dimers and EC-(4β→8)-EC digallate caused an attenuation of NO production paralleled by a decline in MTT reduction of 19% and 55% respectively. EC-(4β→8)-EC caused a decline in MTT reduction which was not observed in single dose treatment (FIG. 3).

### Experiment 2

Test compounds were evaluated with respect to their potential to acutely (24 hour) and chronically (5 doses given in 24 hour intervals) modulate NO production. Positive controls (acetylcholine and/or histamine) and negative control L-NNMA (NO synthase inhibitor) were included in all experiments. Cell counts and total protein were used to assess intra-assay variation. Potential toxic effects of tested compounds were also monitored (MTT reduction was measured).

The data presented herein were obtained from three experiments and were expressed as the concentration of NO present (in μmol/l) (as NOx) in the cell culture medium+/−standard deviation (SD). The data were corrected for the NOx intrinsically present in the fully supplemented cell culture medium and normalized with respect to the volume of media from which the sample was drawn. Data were analyzed using Student's t-test with a 95% level of confidence. P values equal to or less than 0.05 were defined as statistically significant.

The following compounds were tested in vitro:
C-(4α→8)-EC digallate
C-(4α→8)-EC
EC-(4β→8)-C
C-(4β→8)-C
C-(4β→8)-EC
EC-(4β→8)-EC digallate
C-(4β→8)-EC digallate
Epigallocatechingallate (EGCG)
Epicatechingallate (ECG)
Epigallocatechin (EGC)
Gallic Acid

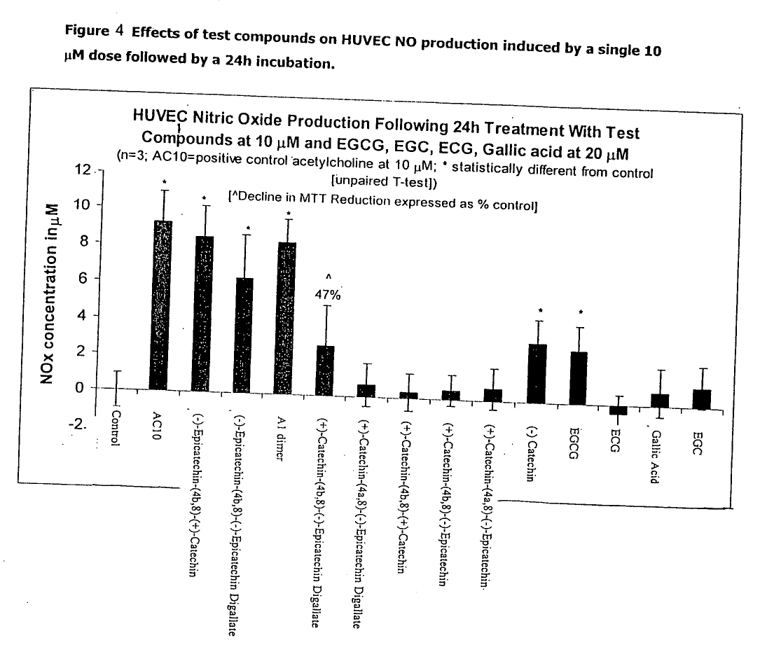

For the acute effect test, HUVECs were incubated with a single dose of the test compounds for 24 hours at concentrations of 100 nM, 1 μM, and 10 μM at 37° C. and 5% CO₂. The positive control, acetylcholine, mediated a statistically significant increase in NOx production as compared to vehicle, and those treated with L-NNMA completely attenuated the acetylcholine-induced NO production. EC-(4β→8)-C, EC-(4β→8)-EC digallate, and Epigallocatechingallate (EGCG) demonstrated a statistically significant ability to modulate NO production when administered at a concentration of 10 μM (FIG. 4).

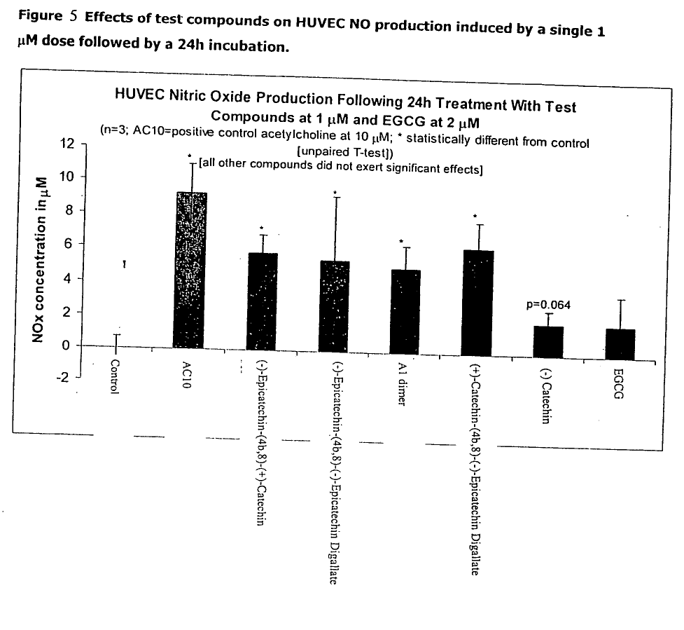

C-(4β→8)-EC digallate caused a 47% loss of HUVEC MTT reduction as compared to controls, when applied at a concentration of 10 μM. EC-(4β→8)-C, EC-(4β→8)-EC digallate, and C-(4β→8)-EC digallate induced statistically significant increases in NO production at a concentration of 1 μM (FIG. 5). At this concentration, there was no effect on cell-mediated reduction of MTT. Other compounds and other concentrations failed to modulate NO synthesis after the 24 hour incubation.

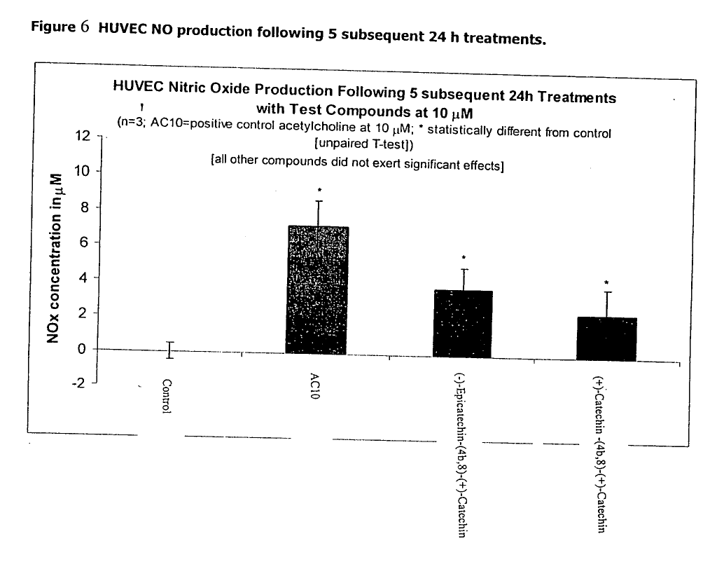

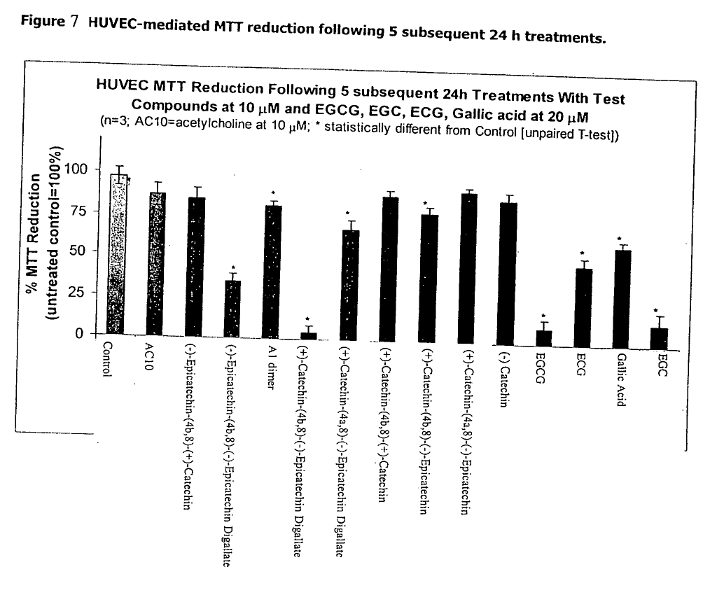

For the chronic effect test, HUVECs were incubated with 5 subsequent doses of test compounds, each for 24 hours. After each 24 hour treatment, culture medium was replaced. C-(4β→8)-C mediated an increase in HUVEC NO production, which was not observed with a single dose. EC-(4β→8)-C caused an increase in NO production which was significantly lower as compared to single dose treatment. The effect of the positive control following the 5 day culture period was also significantly reduced (FIG. 6). EC-(4β→8)-EC digallate, C-(4β→8)-EC digallate, C-(4α→8)-EC digallate, C-(4β→8)-EC, EGCG, ECG, EGC, and gallic acid caused a significant decline of cell-mediated MTT reduction when applied at concentration of 10 μM and 20 μM for the Epicatechin gallates. EC-(4β→8)-EC digallate, C-(4α→8)-EC digallate, EGCG, and EGC also induced a decline in MTT reduction when used at 1 μM or 2 μM for EGC (FIG. 7).

### Ex vivo Experiments

Effect of different procyanidins on endothelium-dependent relaxation was tested in an ex vivo experiment performed as previously described by Karim et al., J. Nutrl Suppl., 130 (8S): 2105S-2108S (2000), the relevant portions of which are hereby incorporated herein by reference. The advantage of using this method is that it assesses functional vascular end points. The method is only able to assess acute events and does not allow for the identification of drug-induced protein expression/activity.

In summary, rabbit aortic rings were obtained from male New Zealand White rabbits. Following isolation, the rings were mounted in oxygenated Kreb's buffer, and pre-constricted with norepinephrine (NE) (10⁻⁶ M). When the tension had reached a steady state, cumulative concentrations of the test compounds were applied (10⁻⁹ to 10⁻⁴ M).

A positive control acetylcholine (10⁻⁹ to 10⁻⁴ M) and a negative control L-NAME were included in the experiment. Use of L-NAME, which is a NO synthase (NOS) inhibitor, allows for differentiating between endothelium dependent and endothelium independent relaxation events. Denuding of aortic rings represent a similar control. 400 U/mL of catalase was added into the aortic bath prior to the addition of each of the test compounds to ensure that the observed effects are not caused by hydrogen peroxide (H₂O₂) generation in the culture medium. The relaxation response was measured as a function of the decrease in the tension (g) exerted by the aortic rings over time using an unpaired Student'T-Test with 95% level of confidence. Data obtained were expressed as a percent relaxation of the norepinephrine (NE) constricted rings. The same statistical approach as described above was used. Dose response curves were obtained by plotting the average percent relaxation (+/−SE) against the concentrations used.

The relaxation responses were attenuated when the vessels were either denuded or pre-treated with L-NAME. Acetylcholine typically produced a relaxation response at a concentration of 10⁻⁷M, reaching maximal relaxation at a concentration of 10⁻⁶M. No response was observed when vessels were treated with vehicle alone.

### Experiment 1

The following produced relation responses as indicated:
EC-(4β→8)-EC digallate (73.55+/−7.08, at 10⁻⁴ M)
EC-(4β→8)-C (58.36+/−11.35, at 10⁻⁴ M)
C-(4α→8)-C (60.80+/−15.09, at 10⁻⁴ M)

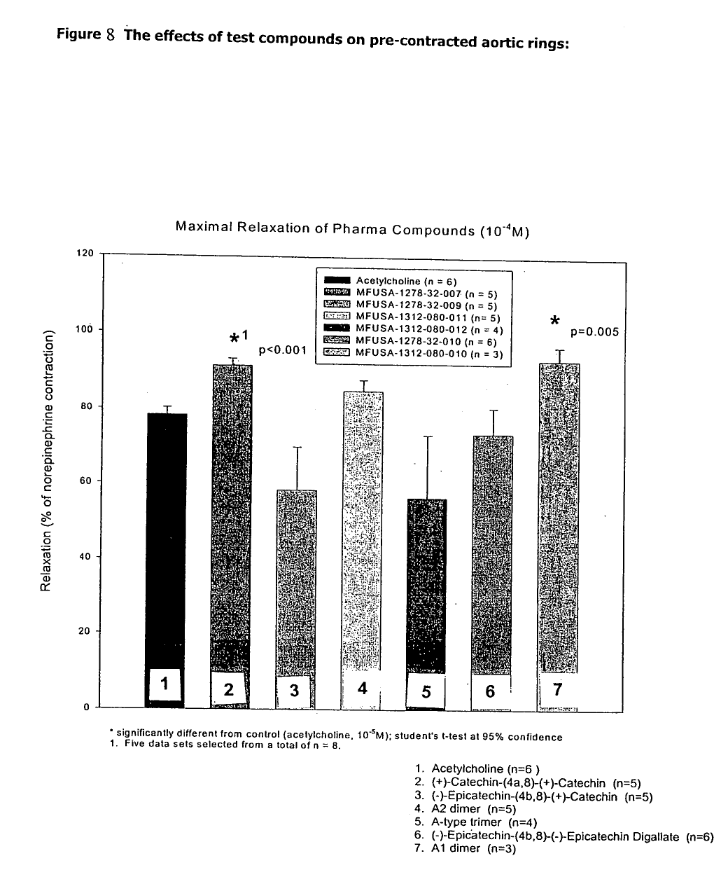

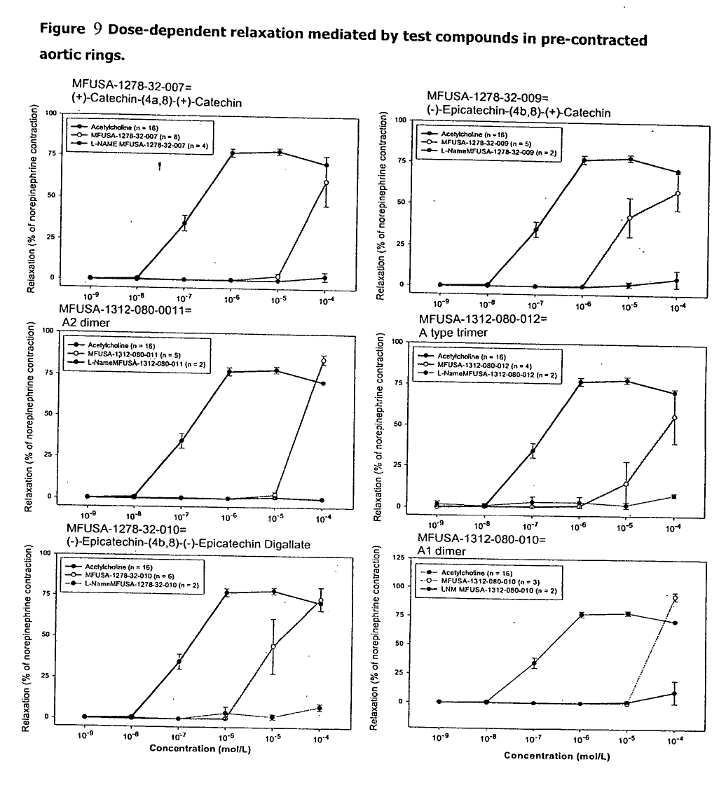

EC-(4β→8)-C and EC-(4β→8)-EC digallate produced a significant relaxation response at concentration of 10⁻⁵ M (42.7+/−11.81; 44.6+/−16.6, respectively). No significant relaxation response was observed for treatment with the EC 4β→8 EC, methylated B2 dimers, EC(4β→8)(0-Methyl)4 EC digallate, or C-(4β→8)-C (FIGS. 8 and 9).

### Experiment 2

The relaxation responses were attenuated when the vessels were either denuded or pre-treated with L-NAME. Acetylcholine typically produced a relaxation response at a concentration of 10⁻⁷M, reaching maximal relaxation at a concentration of 10⁻⁶M. No response was observed when vessels were treated with vehicle alone.

The following produced relation responses as indicated (at 10⁻⁴ M):
C-(4α→8)-C digallate (71.7+/−11.6, at 10⁻⁴ M)
C-(4β→8)-C digallate (72.6+/−9.8, at 10⁻⁴ M)
EC-(4β→8)-C digallate (56.7+/−15.4, at 10⁻⁴ M)
C-(4β→8)-EC digallate (72.4+/−17.4, at 10⁻⁴ M)
C-(4α→8)-EC digallate (40.7+/−9.2, at 10⁻⁴ M)

Additionally, C-(4β→8)-EC digallate also produced a relaxation reponse of 33.1% at 10⁻⁵ M.

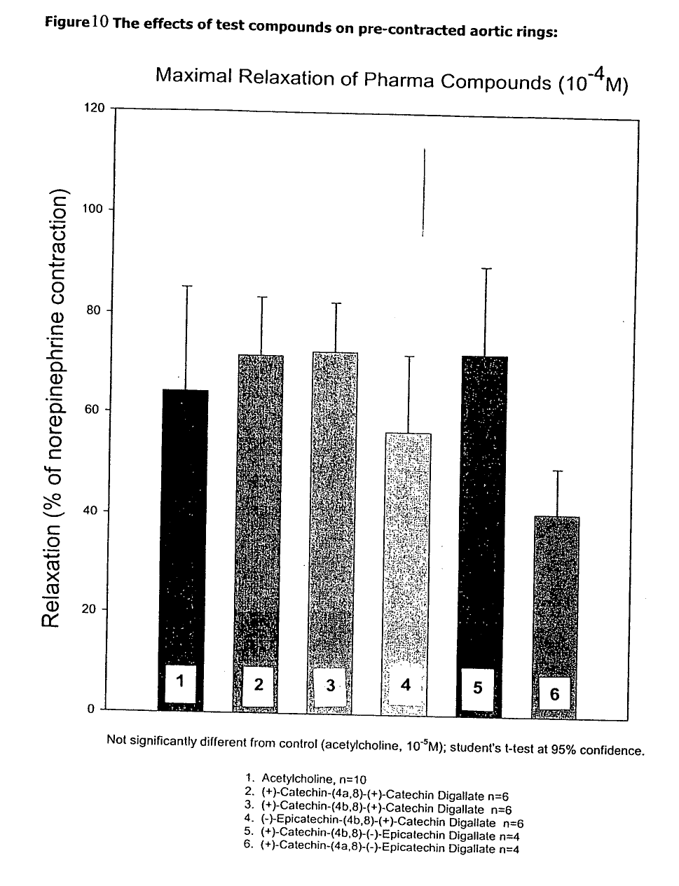

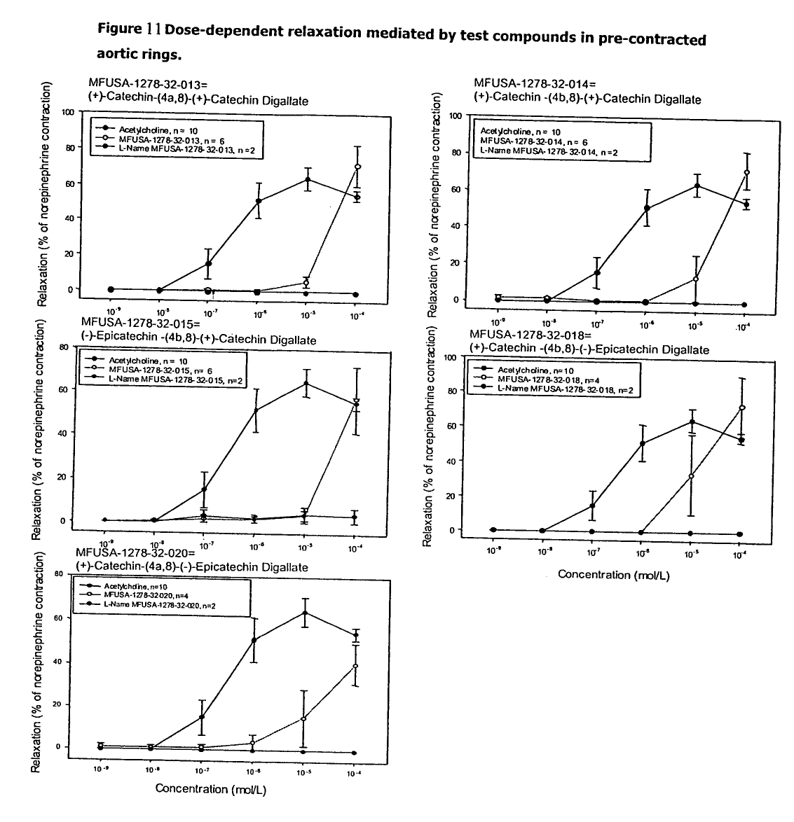

No significant relaxation response was observed for treatment with C-(4β→8)-C, C-(4β→8)-EC, C-(4α→8)-EC and EC(4β→6)EC-8methyl. FIG. 10 compares the maximal relaxation effects of the test compounds (10⁻⁴ M) compared to the maximal relaxation of acetylcholine (10⁻⁵ M, mean +/−SE). FIG. 11 illustrates the dose response curves of acetylcholine, the test compound, and the test compound plus L-Name.

### Experiment 3

The following compounds induced vasorelaxation:

| | Max Relaxation |
|---|---|
| EC(4β→8)EC digallate (10 μM and 100 μM) | 74% |
| EC(4β→8)C digallate (100 μM) | 57% |
| C(4α→8)C digallate (100 μM) | 72% |
| C(4β→8)C digallate (100 μM) | 73% |
| C(4α→8)EC digallate (100 μM) | 41% |
| C(4β→8)EC digallate (10 μM and 100 μM) | 72% |
| C(4α→8)C (100 μM) | 61% |

### Experiment 4

No significant relaxation response was observed for treatment using 10⁻⁹M -10⁴M of Gallic Acid, EGC, ECG or EGCG.

Summary of the above data for selected compounds is represented in Tables 1 and 2.

TABLE 1

| | Aortic Ring | | |
|---|---|---|---|
| Compound | Relax? | Effective Conc, | |
| (+)-Catechin-(4α,8)-(+)-Catechin | Y | 100 | uM |
| (−)-Epicatechin-(4β,8)-(+)-Catechin* | N | — | |
| (−)-Epicatechin-(4β,8)-(−)-Epicatechin | Y | 10 & 100 | uM |
| Digallate | | | |
| (−)-Epicatechin-(4β,8)-(+)-Catechin Digallate | Y | 100 | uM |
| (+)-Catechin-(4α,8)-(+)-Catechin Digallate | Y | 100 | uM |
| (+)-Catechin-(4β8)-(+)-Catechin Digallate | Y | 100 | uM |
| (+)-Catechin-(4β,8)-(−)-Epicatechin Digallate | Y | 10 & 100 | uM |
| (+)-Catechin-(4a,8)-(−)-Epicatechin Digallate | Y | 100 | uM |

| | | |
|---|---|---|
| (+)-Catechin-(4β,8)-(+)-Catechin | N | — |
| (−)-Epicatechin-(4β,8)-(OMethyl)4-(−)- | N | — |
| Epicatechin Digallate | | |
| Epigallocatechingallate (EGCG) | N | — |

*= (−)-Epicatechin-(4β,8)-(+)-Catechin produced significant relaxation in the initial experiments, however, it was subsequently determined that the compounds was contaminated with A1 dimer, and when re-purified, no significant relaxation effect was observed.

TABLE 2

| | Increase in | HUVEC Cells | | |
|---|---|---|---|---|
| Compound | NOx? | 2 hr | 24 hr | 5 d |
| (+)-Catechin-(4α,8)-(+)-Catechin | Y | no effect | 10 uM, sign. NOx incr. | no effect |
| (−)-Epicatechin-(4β,8)-(+)-Catechin* | Y* | 10 uM, sign. NOx | 1 uM and 10 uM, sign. Incr. in | 10 uM, significant NOx |
| | | inc.* | NOx* | increase* |
| (−)-Epicatechin-(4β,8)-(−)-Epicatechin | Y | 10 uM, sign. NOx | 1 and 10 uM, sign. NOx inc. | No significant NOx increase; |
| Digallate | | inc. | | Cell toxicity |
| (−)-Epicatechin-(4β,8)-(+)-Catechin | Not available | Not available | Not available | Not available |
| Digallate | | | | |
| (+)-Catechin-(4α,8)-(+)-Catechin Digallate | Not available | Not available | Not available | Not available |
| (+)-Catechin-(4β,8)-(+)-Catechin Digallate | Not available | Not available | Not available | Not available |
| (+)-Catechin-(4β,8)-(−)-Epicatechin | Y | not tested | 1 uM, sign. Incr. NOx; 10 uM, | No significant NOx increase; |
| Digallate | | | no sign. effect on NOx, Cell | Cell toxicity |
| | | | toxicity | |
| (+)-Catechin-(4α,8)-(−)-Epicatechin | N | not tested | no effect | No significant NOx increase; |
| Digallate | | | | Cell toxicity |
| (+)-Catechin-(4β,8)-(+)-Catechin | Y | no effect | no effect | 10 uM, significant NOx increase |
| (−)-Epicatechin-(4β,8)-(OMethyl)4-(−)- | Y | no effect | no effect | 10 uM, significant NOx increase |
| Epicatechin Digallate | | | | |
| Epigallocatechingallate (EGCG) | Y | not tested | 20 uM, sign. NOx inc. | No significant NOx increase; |
| | | | | Cell toxicity |

*= (−)-Epicatechin-(4β,8)-(+)-Catechin produced significant relaxation in the initial experiments, however, it was subsequently determined that the compounds was contaminated with A1 dimer, and when re-purified, no significant relaxation effect was observed.

### Example 4

Determination of Flavanols/Procyanidins

Procyanidins were quantified as follows: a composite standard was made using commercially available (−)-epicatechin, and dimers through decamers obtained in a purified state by the methods described in Hammerstone, J. F. et al., J. Ag. Food Chem.; 1999; 47 (10) 490-496; Lazarus, S. A. et al., J. Ag. Food Chem.; 1999; 47 (9); 3693-3701; and Adamson, G. E. et al., J. Ag. Food Chem.; 1999; 47 (10) 4184-4188. Standard Stock solutions using these compounds were analyzed using the normal-phase HPLC method described in the previously cited Adamson reference, with fluorescence detection at excitation and emission wavelengths of 276 nm and 316 nm, respectively. Peaks were grouped and their areas summed to include contributions from all isomers within any one class of oligomers and calibration curves were generated using a quadratic fit. Monomers and smaller oligomers had almost linear plots which is consistent with prior usage of linear regression to generate monomer-based and dimer-based calibration curves.

These calibration curves were then used to calculate procyanidin levels in samples prepared as follows: First, the cocoa or chocolate sample (about 8 grams) was defatted using three hexane extractions (45 mL each). Next, one gram of defatted material was extracted with 5 mL of the acetone/water/acetic acid mixture (70:29.5:0.5 v/v). The quantity of procyanidins in the defatted material was then determined by comparing the HPLC data from the samples with the calibration curves obtained as described above (which used the purified oligomers). The percentage of fat for the samples (using a one gram sample size for chocolate or one-half gram sample size for liquors) was determined using a standardized method by the Association of Official Analytical Chemists (AOAC Official Method 920.177). The quantity of total procyanidin levels in the original sample (with fat) was then calculated. Calibration was performed prior to each sample run to protect against column-to-column variations.

1. A pharmaceutical composition comprising a therapeutically effective amount of EC-(4β→8)-C digallate.

2. The pharmaceutical composition of claim 1 wherein EC-(4β→8)-C digallate is (−)EC-(4β→8)-(+)C digallate.

3. The pharmaceutical composition of claim 1 further comprising an additional NO modulating agent and/or a vascular-protective or therapeutic agent.

4. A pharmaceutical composition comprising a therapeutically effective amount of C-(4α→8)-C digallate.

5. The pharmaceutical composition of claim 4 wherein C-(4α→8)-C digallate is (+)C-(4α→8)-(+)C digallate.

6. The composition of claim 4 further comprising an additional NO modulating agent and/or a vascular-protective or therapeutic agent.

7. A pharmaceutical composition comprising a therapeutically effective amount of C-(4β→8)-C digallate.

8. The pharmaceutical composition of claim 7 wherein C-(4β→8)-C digallate is (+)C-(4β→8)-(+)C digallate.

9. The composition of claim 7 further comprising an additional NO modulating agent and/or a vascular-protective or therapeutic agent.

10. A pharmaceutical composition comprising a therapeutically effective amount of C-(4β→8)-EC digallate.

11. The pharmaceutical composition of claim 10 wherein C-(4β→8)-EC digallate is (+)C-(4β→8)-(−)EC digallate.

12. The composition of claim 10 further comprising an additional NO modulating agent and/or a vascular-protective or therapeutic agent.

13. A pharmaceutical composition comprising a therapeutically effective amount of a dimer comprising an alpha linkage, for example C-(4α→8)-C.

14. The pharmaceutical composition of claim 13 wherein C-(4α→8)-C is (+)C-(4α→8)-(+)C.

15. The composition of claim 13 further comprising an additional NO modulating agent and/or a vascular-protective or therapeutic agent.

16. A method of treating or preventing an NO-responsive disease or disorder by administering to a subject in need thereof an effective amount of EC-(4β→8)-C digallate, or a pharmaceutically acceptable salt or derivative thereof, wherein the subject is a human or a veterinary animal.

17. The method of claim 16, wherein the subject is a human.

18. The method of claim 17, wherein EC-(4β→8)-C digallate is (−)EC-(4β→8)-(+)C digallate.

19. The method of claim 16, wherein the NO-responsive disease or disorder is hypertension, cardiovascular disease, coronary artery disease, vascular circulation disorders, diabetes, cognitive dysfunction or disorder and/or renal disease.

20. The method of claim 16, wherein the subject is at risk of heart attack, stroke, congestive heart failure, and/or kidney failure.

21. The method of claim 16, wherein the subject suffers from high blood pressure in combination with any of the following: diabetes, obesity, adverse lipid profile and/or smoking.

22. A method of treating or preventing an NO-responsive disease or disorder by administering to a subject in need thereof an effective amount of C-(4α→8)-C digallate or a pharmaceutically acceptable salt or derivative thereof, wherein the subject is a human or a veterinary animal.

23. The method of claim 22 wherein the subject is a human.

24. The method of claim 23 wherein C-(4α→8)-C digallate is (+)C-(4α→8)-(+)C digallate.

25. The method of claim 22, wherein the NO-responsive disease or disorder is hypertension, cardiovascular disease, coronary artery disease, vascular circulation disorders, diabetes, cognitive dysfunction or disorder and/or renal disease.

26. The method of claim 22, wherein the subject is at risk of heart attack, stroke, congestive heart failure, and/or kidney failure.

27. The method of claim 22, wherein the subject suffers from high blood pressure in combination with any of the following: diabetes, obesity, adverse lipid profile and/or smoking.

28. A method of treating or preventing an NO-responsive disease or disorder by administering to a subject in need thereof an effective amount of C-(4β→8)-C digallate or a pharmaceutically acceptable salt or derivative thereof, wherein the subject is a human or a veterinary animal.

29. The method of claim 28 wherein the subject is a human.

30. The method of claim 29 wherein C-(4β→8)-C digallate is (+)C-(4β→8)-(+)C digallate.

31. The method of claim 28, wherein the NO-responsive disease or disorder is hypertension, cardiovascular disease, coronary artery disease, vascular circulation disorders, diabetes, cognitive dysfunction or disorder and/or renal disease.

32. The method of claim 28, wherein the subject is at risk of heart attack, stroke, congestive heart failure, and/or kidney failure.

33. The method of claim 28, wherein the subject suffers from high blood pressure in combination with any of the following: diabetes, obesity, adverse lipid profile and/or smoking.

34. A method of treating or preventing an NO-responsive disease or disorder by administering to a subject in need thereof an effective amount of C-(4β→8)-EC digallate or a pharmaceutically acceptable salt or derivative thereof, wherein the subject is a human or a veterinary animal.

35. The method of claim 34 wherein the subject is a human.

36. The method of claim 35 wherein C-(4β→8)-EC digallate is (+)C-(4β→8)-(−) EC digallate.

37. The method of claim 34, wherein the NO-responsive disease or disorder is hypertension, cardiovascular disease, coronary artery disease, vascular circulation disorders, diabetes, cognitive dysfunction or disorder and/or renal disease.

38. The method of claim 34, wherein the subject is at risk of heart attack, stroke, congestive heart failure, and/or kidney failure.

39. The method of claim 34, wherein the subject suffers from high blood pressure in combination with any of the following: diabetes, obesity, adverse lipid profile and/or smoking.

40. A method of treating or preventing an NO-responsive disease or disorder by administering to a subject in need thereof an effective amount of a dimer comprising an alpha linkage, for example C-(4α→8)-C or a pharmaceutically acceptable salt or derivative thereof, wherein the subject is a human or a veterinary animal.

41. The method of claim 40 wherein the subject is a human.

42. The method of claim 41 wherein C-(4α→8)-C is (+)C-(4α→8)-(+)C.

43. The method of claim 40, wherein the NO-responsive disease or disorder is hypertension, cardiovascular disease, coronary artery disease, vascular circulation disorders, diabetes, cognitive dysfunction or disorder and/or renal disease.

44. The method of claim 40, wherein the subject is at risk of heart attack, stroke, congestive heart failure, and/or kidney failure.

45. The method of claim 40, wherein the subject suffers from high blood pressure in combination with any of the following: diabetes, obesity, adverse lipid profile and/or smoking.

46. A method of treating hypertension by administering to a subject in need thereof an effective amount of EC-(4β→8)-C digallate, or a pharmaceutically acceptable salt or derivative thereof, wherein the subject is a human or a veterinary animal.

47. The method of claim 46 wherein the subject is a human.

48. The method of claim 47 wherein EC-(4β→8)-C digallate is (−)EC-(4β→8)-(+)C digallate.

49. The method of claim 46, wherein the subject suffers from high blood pressure in combination with any of the following: diabetes, obesity, adverse lipid profile and/or smoking.

50. A method of treating hypertension by administering to a subject in need thereof an effective amount of C-(4α→8)-C digallate or a pharmaceutically acceptable salt or derivative thereof, wherein the subject is a human or a veterinary animal.

51. The method of claim 50 wherein the subject is a human.

52. The method of claim 51 wherein C-(4α→8)-C digallate is (+)C-(4α→8)-(+)C digallate.

53. The method of claim 50, wherein the subject suffers from high blood pressure in combination with any of the following: diabetes, obesity, adverse lipid profile and/or smoking.

54. A method of treating hypertension by administering to a subject in need thereof an effective amount of C-(4β→8)-C digallate or a pharmaceutically acceptable salt or derivative thereof, wherein the subject is a human or a veterinary animal.

55. The method of claim 54 wherein the subject is a human.

56. The method of claim 55 wherein C-(4β→8)-C digallate is (+)C-(4β→8)-(+)C digallate.

57. The method of claim 54, wherein the subject suffers from high blood pressure in combination with any of the following: diabetes, obesity, and/or smoking.

58. A method of treating hypertension by administering to a subject in need thereof an effective amount of C-(4β→8)-EC digallate or a pharmaceutically acceptable salt or derivative thereof, wherein the subject is a human or a veterinary animal.

59. The method of claim 58 wherein the subject is a human.

60. The method of claim 59 wherein C-(4β→8)-EC digallate is (+)C-(4β→8)-(−) EC digallate.

61. The method of claim 58, wherein the subject suffers from high blood pressure in combination with any of the following: diabetes, obesity, and/or smoking.

62. A method of treating hypertension by administering to a subject in need thereof an effective amount of a dimer comprising an alpha linkage, for example, C-(4α→8)-C or a pharmaceutically acceptable salt or derivative thereof, wherein the subject is a human or a veterinary animal.

63. The method of claim 58 wherein the subject is a human.

64. The method of claim 59 wherein C-(4α→8)-C is (+)C-(4α→8)-(+)C.

65. The method of claim 58, wherein the subject suffers from high blood pressure in combination with any of the following: diabetes, obesity, and/or smoking.
